SEQUENCE LISTING (69 sequences)

SEQ ID NO: 1 - DNA, 546 bp, Bacillus subtilis
  CDS (1)..(546)
atgaaaaaatgtcttctatttctaacaaccattgcacttattctgtcattaagcacaaatgcatttgcgaaaaatacatcaggcgatttatcacaaaaacaagcattacagttggcattatccgctagagaacatttttggaacaccatgagcggccacaatccgaaggtgaaaaaagcagtttgcccatcaggcacattcgagtatcaaaatcttcaatatgtatacatgtgcagtgatctaggcactaaagcaaaagcggtgaattatttaactcctatttttacaaaaacagcaatcgaaaaaggctttaaagattatcatttcactgtttcaaaaggaaaacttgctgttccaatcggtgacggagacaatcttttgaattggaaaaaatcaactgctaaactgatctctaaaaaaggcagcacaatcacatacgaatttactgtccctacattagacggatccccttctgcaaaacggaaagtgacatttgtgaaagaaaataaaaaatggaaagtcaatcaatttgacgcagttatataa

SEQ ID NO: 2 - protein, 181 aa, Bacillus subtilis
MKKCLLFLTTIALILSLSTNAFAKNTSGDLSQKQALQLALSAREHFWNTMSGHNPKVKKAVCPSGTFEYQNLQYVYMCSDLGTKAKAVNYLTPIFTKTAIEKGFKDYHFTVSKGKLAVPIGDGDNLLNWKKSTAKLISKKGSTITYEFTVPTLDGSPSAKRKVTFVKENKKWKVNQFDAVI

SEQ ID NO: 3 - DNA, 1467 bp, Bacillus subtilis
  CDS (1)..(1467)
atgaaaaagaaattagcagcagggctgacagcatctgcgattgtcggcacaactttagtagtgacaccagctgaagcagcaacgattaaggtcaaaagcggagattctttatggaaactggcccaaacctataacacaagtgtggcggccctcacatccgccaatcatctttcgactactgttctgtcgatcggccagacgcttactattccaggcagcaaatccagcacttcttcatctacttcaagctcaacaaccaagaagagcggaagttctgtttacacagtgaaaagcggagattcactttggctgatcgcaaatgagtttaaaatgacggtgcaggaactgaaaaaattaaacggactaagcagcgatttaattcgtgctgggcagaaattaaaggtgtccggtactgtttcttcaagctctagttccagtaaaaaaagcaactctaataagagttcaagctccagttcaaaaagcagttctaataagagttcaagttcttcatcttctacgggaacatataaggtgcagcttggagattcactttggaaaattgcaaacaaagtcaatatgtccatcgctgaattgaaggtcttgaacaacttaaaatcagacaccatttacgtgaatcaggtgttgaaaacaaaatcaagcggctctgatacgtcttctaaagacaattcatccaaatcgaaccaaacttcagcaacaactaaatataccgttaaaagcggtgattcactttggaaaatcgcaaacaactataacctgactgtacagcaaatccgaaatatcaacaatctgaaatcagatgtgctttacgttggacaagtattgaagctgacaggtaaagcatcttcaggctcttcatcatcgtcgtcttcttcgtcaaatgcaagctccggcacgacgactacatacacggtgaaaagcggtgattccttatgggtgattgctcaaaaatttaatgtaacagctcagcagattcgtgagaaaaacaatttaaaaacggatgtcttgcaggttggccaaaagctggtcatttccggaaaagcttcatcatcgtcttcatccggttcttcaaacaccaccagctcaacgagtgcgaagattaacacaatgatttcggccgctaaagcgcagcttggggttccgtatcgctggggaggcacgacaccgtccgggtttgactgcagcggattcatttactatgtactgaacaaagtcacatccgtatctagattaacggcagcgggatattggaacacaatgaaatcagttagccagcctgctgtcggagatttcgttttcttttcaacatataaagccggcccttcccacgtcggaatttacctaggcaacggggaattcataaatgccaacgattccggagttgtcatttccaatatgaacaactcttactggaaacagcgctatctcggtgcaaaacgatatttctaa

SEQ ID NO: 4 - protein, 488 aa, Bacillus subtilis
MKKKLAAGLTASAIVGTTLVVTPAEAATIKVKSGDSLWKLAQTYNTSVAALTSANHLSTTVLSIGQTLTIPGSKSSTSSSTSSSTTKKSGSSVYTVKSGDSLWLIANEFKMTVQELKKLNGLSSDLIRAGQKLKVSGTVSSSSSSSKKSNSNKSSSSSSKSSSNKSSSSSSSTGTYKVQLGDSLWKIANKVNMSIAELKVLNNLKSDTIYVNQVLKTKSSGSDTSSKDNSSKSNQTSATTKYTVKSGDSLWKIANNYNLTVQQIRNINNLKSDVLYVGQVLKLTGKASSGSSSSSSSSSNASSGTTTTYTVKSGDSLWVIAQKFNVTAQQIREKNNLKTDVLQVGQKLVISGKASSSSSSGSSNTTSSTSAKINTMISAAKAQLGVPYRWGGTTPSGFDCSGFIYYVLNKVTSVSRLTAAGYWNTMKSVSQPAVGDFVFFSTYKAGPSHVGIYLGNGEFINANDSGVVISNMNNSYWKQRYLGAKRYF

SEQ ID NO: 5 - DNA, 1005 bp, Bacillus subtilis
  CDS (1)..(1005)
atgaaaaagcaaatcattacagctacgacagcagttgttttaggatcgacgttatttgcaggagcggcatctgcacaaagcattaaggtgaaaaaaggcgacacgttatgggatctttcaagaaaatacgacacaacgatcagtaaaattaaatcagagaaccaccttcgttcagacattatttatgtgggacaaactttatcgattaacggcaaatctacaagttcaaaaagcagcagttcttcttcctcttcttctacatacaaagtaaagagcggggacagcctttggaaaatttcaaaaaaatacggcatgacaatcaatgaactgaagaagctgaatggcttaaaatcagatttgcttcgtgttggacaagtcctgaaactgaaaggttcaactagttcaagcagctccagctcatcaaaagtgtcatcgtcttcaacttctacttataaagtgaagagcggagacagcctttctaaaattgcgagcaaatacggcactacggttagcaaattaaaaagcttaaacggcttaaaatcagatgtaatctatgtcaaccaagtattgaaggtgaaaggaacaagcacaagcagctcaaaacctgcttcatcttcatcgtcttcaagcagcaaaacgtcatctacatcacttaatgtgagcaagctggtttctgatgcaaaagcgttagtcggaacgccatataaatggggcggaacgacaacttcaggctttgactgcagcggattcatttggtacgtactgaataaacaaacaagtgtgggcagaacaagcactgcaggatactggagttctatgaagagcattgccagcccgtctgttggtgatttcgtcttcttcacaacatataaatccggcccttctcacatggggatttacattggaaacaacagtttcattcatgcaggatctgacggcgtacaaatcagcagcctgaacaacagctactggaagcctcgttacctcggtgcgaaaagattctaa

SEQ ID NO: 6 - protein, 334 aa, Bacillus subtilis
MKKQIITATTAVVLGSTLFAGAASAQSIKVKKGDTLWDLSRKYDTTISKIKSENHLRSDIIYVGQTLSINGKSTSSKSSSSSSSSSTYKVKSGDSLWKISKKYGMTINELKKLNGLKSDLLRVGQVLKLKGSTSSSSSSSSKVSSSSTSTYKVKSGDSLSKIASKYGTTVSKLKSLNGLKSDVIYVNQVLKVKGTSTSSSKPASSSSSSSSKTSSTSLNVSKLVSDAKALVGTPYKWGGTTTSGFDCSGFIWYVLNKQTSVGRTSTAGYWSSMKSIASPSVGDFVFFTTYKSGPSHMGIYIGNNSFIHAGSDGVQISSLNNSYWKPRYLGAKRF

SEQ ID NO: 7 - DNA, 18 bp, Artificial
  Oligonucleotide as PCR primer eprfw1
ctccggcagaaagggcat

SEQ ID NO: 8 - DNA, 32 bp, Artificial
  Oligonucleotide as PCR primer eprUPr
cgttcagccgtacaacaagtttgcaagacatg

SEQ ID NO: 9 - DNA, 31 bp, Artificial
  Oligonucleotide as PCR primer eprDNf
aacttgttgtacggctgaacgccgtcaaacc

SEQ ID NO: 10 - DNA, 39 bp, Artificial
  Oligonucleotide as PCR primer eprrv-repU
ctgatctcgacttcgttcagacgggtcgtacaatggctg

SEQ ID NO: 11 - DNA, 18 bp, Artificial
  Oligonucleotide as PCR primer repUfw
gaacgaagtcgagatcag

SEQ ID NO: 12 - DNA, 21 bp, Artificial
  Oligonucleotide as PCR primer Cmrv1
gctgtaatataaaaaccttct

SEQ ID NO: 13 - DNA, 18 bp, Artificial
  Oligonucleotide as PCR primer eprfw2
cgacaccatagctttctg

SEQ ID NO: 14 - DNA, 18 bp, Artificial
  Oligonucleotide as PCR primer Cmrv2
aactaacggggcaggtta

SEQ ID NO: 15 - DNA, 41 bp, Artificial
  Oligonucleotide as PCR primer repUr-CmU
agaatcaatcccatggtctcacttttccactttttgtcttg

SEQ ID NO: 16 - DNA, 41 bp, Artificial
  Oligonucleotide as PCR primer CmUf-repU
gtgagaccatgggattgattctaatgaagaaagcagacaag

SEQ ID NO: 17 - DNA, 20 bp, Artificial
  Oligonucleotide as PCR primer wprAfw1
gggtaatttatctgataggg

SEQ ID NO: 18 - DNA, 33 bp, Artificial
  Oligonucleotide as PCR primer wprAUPr
cttttgcttcccacaaccgagctgaattttctg

SEQ ID NO: 19 - DNA, 34 bp, Artificial
  Oligonucleotide as PCR primer wprADNf
ctcggttgtgggaagcaaaagttgttgttgaaaa

SEQ ID NO: 20 - DNA, 39 bp, Artificial
  Oligonucleotide as PCR primer wprArv-repU
ctgatctcgacttcgttcatcctcattgaagacggcatc

SEQ ID NO: 21 - DNA, 20 bp, Artificial
  Oligonucleotide as PCR primer wprAfw2
ggaacatatatgacacacct

SEQ ID NO: 22 - DNA, 19 bp, Artificial
  Oligonucleotide as PCR primer mprfw1
tgtttggtgttgagctgtt

SEQ ID NO: 23 - DNA, 34 bp, Artificial
  Oligonucleotide as PCR primer mprUPr
ttcgtgtgaatccattgttttctgaatcttggaa

SEQ ID NO: 24 - DNA, 33 bp, Artificial
  Oligonucleotide as PCR primer mprDNf
gaaaacaatggattcacacgaacggaggatcgt

SEQ ID NO: 25 - DNA, 42 bp, Artificial
  Oligonucleotide as PCR primer mprrv-repU
ctgatctcgacttcgttcctcgtaagaaaaaatacctatttc

SEQ ID NO: 26 - DNA, 19 bp, Artificial
  Oligonucleotide as PCR primer mprfw2
ccttgcgaaagatagggta

SEQ ID NO: 27 - DNA, 18 bp, Artificial
  Oligonucleotide as PCR primer nprBfw1
tttttagcagtggtgctc

SEQ ID NO: 28 - DNA, 32 bp, Artificial
  Oligonucleotide as PCR primer nprBUPr
catacgccttcagtaatagagatgtcttggtc

SEQ ID NO: 29 - DNA, 32 bp, Artificial
  Oligonucleotide as PCR primer nprBDNf
ctctattactgaaggcgtatgaggctgtcggc

SEQ ID NO: 30 - DNA, 40 bp, Artificial
  Oligonucleotide as PCR primer nprBrv-repU
ctgatctcgacttcgttcttccaaatgcgcttcattagga

SEQ ID NO: 31 - DNA, 18 bp, Artificial
  Oligonucleotide as PCR primer nprBfw2
cttttgagcccgttcctc

SEQ ID NO: 32 - DNA, 19 bp, Artificial
  Oligonucleotide as PCR primer bprfw1
tgagattgtggtgacagtg

SEQ ID NO: 33 - DNA, 33 bp, Artificial
  Oligonucleotide as PCR primer bprUPr
ttaagttttccgctgatgagtctgtttttcgtt

SEQ ID NO: 34 - DNA, 35 bp, Artificial
  Oligonucleotide as PCR primer bprDNf
gactcatcagcggaaaacttaatatgaacacagaa

SEQ ID NO: 35 - DNA, 41 bp, Artificial
  Oligonucleotide as PCR primer bprrv-repU
ctgatctcgacttcgttcgtaatcatgacaccgttttgaac

SEQ ID NO: 36 - DNA, 19 bp, Artificial
  Oligonucleotide as PCR primer bprfw2
attgcaaccggctttatcg

SEQ ID NO: 37 - DNA, 19 bp, Artificial
  Oligonucleotide as PCR primer nprEfw1
ttttgaagacgttcggcga

SEQ ID NO: 38 - DNA, 34 bp, Artificial
  Oligonucleotide as PCR primer nprEUPr
attgtctgttgctgaagcagcctggaatgctgtt

SEQ ID NO: 39 - DNA, 34 bp, Artificial
  Oligonucleotide as PCR primer nprEDNf
caggctgcttcagcaacagacaatttcttaccta

SEQ ID NO: 40 - DNA, 38 bp, Artificial
  Oligonucleotide as PCR primer nprErv-repU
ctgatctcgacttcgttccctctcttaagtaagcgctg

SEQ ID NO: 41 - DNA, 21 bp, Artificial
  Oligonucleotide as PCR primer nprEfw2
tctctttgttgaaaaacgata

SEQ ID NO: 42 - DNA, 19 bp, Artificial
  Oligonucleotide as PCR primer vprfw1
aaaaacatccctccgcttc

SEQ ID NO: 43 - DNA, 32 bp, Artificial
  Oligonucleotide as PCR primer vprUPr
tcttcggtcaatacaagcagaaagcgaatgat

SEQ ID NO: 44 - DNA, 32 bp, Artificial
  Oligonucleotide as PCR primer vprDNf
tctgcttgtattgaccgaagaacctttcactg

SEQ ID NO: 45 - DNA, 39 bp, Artificial
  Oligonucleotide as PCR primer vprrv-repU
ctgatctcgacttcgttctgctcggctcatctggagaaa

SEQ ID NO: 46 - DNA, 18 bp, Artificial
  Oligonucleotide as PCR primer vprfw2
tttttggcaggcagcctt

SEQ ID NO: 47 - DNA, 21 bp, Artificial
  Oligonucleotide as PCR primer aprEfw1
ctgtttattatgggccacgaa

SEQ ID NO: 48 - DNA, 32 bp, Artificial
  Oligonucleotide as PCR primer aprEUPr
gatcagcttggggttaatcaacgtacaagcag

SEQ ID NO: 49 - DNA, 33 bp, Artificial
  Oligonucleotide as PCR primer aprEDNf
tgattaaccccaagctgatccacaattttttgc

SEQ ID NO: 50 - DNA, 39 bp, Artificial
  Oligonucleotide as PCR primer aprErv-repu
ctgatctcgacttcgttctgattttccaaacgagctttc

SEQ ID NO: 51 - DNA, 21 bp, Artificial
  Oligonucleotide as PCR primer aprEfw2
atgggccattatgtcatgaag

SEQ ID NO: 52 - DNA, 38 bp, Artificial
  Oligonucleotide as PCR primer yoeBSal-F-2
gcgcagatctgtcgactaagagaaagagattttgaagg

SEQ ID NO: 53 - DNA, 31 bp, Artificial
  Oligonucleotide as PCR primer yoeBSph-R
gcgcgcatgcttatataactgcgtcaaattg

SEQ ID NO: 54 - DNA, 1245 bp, Bacillus subtilis
  CDS (1)..(1245)
atgaaaaagaagattgtagccggcttggctgtttctgcagttgttgggtcgtcgatggccgcagcacccgcggaagcaaaaacgataaaagtaaaaagcggtgactctctttggaaactttcccgccagtatgatacgacaatatcagctctgaaatccgaaaacaaattgaaatcaacggtgctttatgtcggacaaagtcttaaggttcctgaaagcagcaaaaaaagcacgacatccccatctaattcaagcaaaacatcaacatacacagttgcatatggtgattccctatggatgattgctaaaaatcataaaatgagtgtttctgagcttaaaagcttaaacagcctgagcagtgacctgattcgtccggggcaaaagcttaagatcaaagggacaagcagttcaagcggatcaaatggatcaaagaaaaacagcggttccaattcttcaggttcttcaaaaagtacatatacggtcaagcttggtgactcactgtggaaaatcgcaaacagcttaaatatgactgtagccgaattaaaaaccttaaatggcttaacatcagatacactttatcctaaacaggtgctcaaaataaaaggaagcagttcacctaagaacggaaacagcggttccaaaaagccgtcaaactccaatccctcaaaaacaacgacgtacaaggtaaaagcaggcgattcgctgtggaaaattgcaaatcggctcggagtcactgtccaaagcatccgtgataaaaacaatttatcatcggatgtgttgcaaatcgggcaggttctgactatttcgggagcatcaaaatctaatccttcaaatccgacaaagccaacaaagccaaaagataactcagggtcaaacattcaaataggttcgaagattgacagaatgataacggaagcgaaaaaatatgtcggagtgccttaccgatggggcggaaacacgcctgccggttttgattgcagcggcttcatttattacctcattaataatgtatcatctatatcaagattgagtacggcagggtattggaatgtcatgcagaaagtcagccagccgtcagtaggagactttgtcttctttaccacatataaatccggcccgtctcacatggggatttatctaggcggcggtgattttattcatgcaagctcatcgggcgtcgacatttccaatctcagcaattcgtattggaaacagcggtacttgggcgcaagaagttatttttaa

SEQ ID NO: 55 - protein, 414 aa, Bacillus subtilis [sequence omitted]

SEQ ID NO: 56 - DNA, 1422 bp, Bacillus subtilis
  CDS (1)..(1422)
gtgagaaagagtttaattacacttggtttggcttccgtcatcgggacaagcagttttttgatcccatttacaagtaaaactgcatcggcggaaacattagatgaaaagaaacaaaaaatcgaaagcaagcaatctgaggttgcttccagcattgaagcgaaggaaaaagaattaaccgagcttcaggaaaatcaatcaaagattgaaaaagaactgaaagacattaacgataaggcgcttgatacaagcaacaagattgaagataaaaaagaagaaaacgataaaacaaaagaagaaatcaaaaaactgaaaaaagagattaaagaaactgaagcacgcatcgaaaaacgcaatgaaatcctgaaaaaacgcgttcgttctttacaggaaagcggcggatctcaaggatacatagatgttcttttaggatcaacaagctttggtgactttatctctcgtgcgactgcggtttcatctattgtggacgcagacaaagatttaatcaaacagcaagagcaggataaagcgaagctcgaagattctgaagcggatttgaatgacaagctgaaagaagttcaagctgccttggctaaattagaaacaatgcaaaaagaccttgataaacagcttaatgaaaaagacaagcttttcgacgaagcaaaagcaagccaaaagaaaacggctaaagcgatttctgaattaaaatcagaggcgtctgaacttgcaaatcaaaaagcaaatactgaagctgaacaagcacgcatcaaaaaagaacaagaagcagcggctgctttgatcaaaaagcaggaagaagcacaaaaagcatctgatgaaacacaaacagatgacagtcaaacggcgacaactgaatcatcaaaagcaagctcatctgatgattcttcagacaattcttcagacaattcttctaacggttcatcaaacagttcgtcaaacggctcatcttctaagaagagcagcggctcaaacagcaattcaggcggcactgttatcagcaactctggcggaattgaaggcgcgatcagcgttggttcaagcattgtaggacaatctccgtacaagtttggcggcggacgcactcagtctgatatcaacaaccgtatttttgactgctcatcattcgtacgctgggcatacgcttctgcaggtgttaaccttggacctgtaggcggaacaacaactgatacgttagttggcagaggacaagctgtcagcgcgtctgaaatgaaacgcggagaccttgtgttctttgacacttacaaaacaaatggacacgtaggaatctacttaggaaacggtactttcctaaacgacaatacatctcatggcgtatctgttgactctatgagcaatccttactggaaagcagcatttaaaggtgttgtaagacgtgttgttcaataa

SEQ ID NO: 57 - protein, 473 aa, Bacillus subtilis
VRKSLITLGLASVIGTSSFLIPFTSKTASAETLDEKKQKIESKQSEVASSIEAKEKELTELQENQSKIEKELKDINDKALDTSNKIEDKKEENDKTKEEIKKLKKEIKETEARIEKRNEILKKRVRSLQESGGSQGYIDVLLGSTSFGDFISRATAVSSIVDADKDLIKQQEQDKAKLEDSEADLNDKLKEVQAALAKLETMQKDLDKQLNEKDKLFDEAKASQKKTAKAISELKSEASELANQKANTEAEQARIKKEQEAAAALIKKQEEAQKASDETQTDDSQTATTESSKASSSDDSSDNSSDNSSNGSSNSSSNGSSSKKSSGSNSNSGGTVISNSGGIEGAISVGSSIVGQSPYKFGGGRTQSDINNRIFDCSSFVRWAYASAGVNLGPVGGTTTDTLVGRGQAVSASEMKRGDLVFFDTYKTNGHVGIYLGNGTFLNDNTSHGVSVDSMSNPYWKAAFKGVVRRVVQ

SEQ ID NO: 58 - DNA, 990 bp, Bacillus subtilis
  CDS (1)..(990)
atgataagtaaaaaggtagttcttcctttggtattctcggctccttttatcttcttttttgttttatgtatcgtagtggttatgacgatttcaagagaaaatcaagtcggggacgattttataggcggcggggatggcgaatatgaaacagttggaattgcgccggaagtggaacgatttagagcagtatttgagaaatatgcccgtcaagaaggcgtttttgaccaagtgaatattattatggcgctcaccatgcaagaaagtggtgggcgttctttagatatcatgcaaagttccgaaagtatcggcttacctcccaactccataactgacccagaacgttcaattgaagtcggcattaaacattttaagaaggtttttaaacaggctggcggtgatgttcgtttaaccttacaggcatacaactttggttctggatttatagactatgtcaaaaagaatggcgggaaatacacaaagaaattagcacttgatttcagccgattgcaagcttttaaaatgggctggaaaagctacggtgacccaagttatgttgaccatgtcatgaggtatgtgaaaggctctgataaaaatgtgaagcctgtgaaaggctctatggatttctatgaaacggtcatgaaagaagctttgaaatatgaagggcaaccgtatgcctggggaggttcaaacccggaaactggatttgattgttctggccttgttcagtggtcttttgcgaaagctggaatcactcttcctagaacagcacaagaacaacatggggcaaccaagaaaataagtgaaaaagaagcgactgcgggtgacttagtgttctttggcggcacatacgaaggaaaagccattactcacgtcggcatctatgtaggcaatggcagaatgtttaattcaaatgacagtggaatccaatattctgatctgaaatcaggttactggagagatcaccttgtatcttttggacgcattaaataa

SEQ ID NO: 59 - protein, 329 aa, Bacillus subtilis
MISKKVVLPLVFSAPFIFFFVLCIVVVMTISRENQVGDDFIGGGDGEYETVGIAPEVERFRAVFEKYARQEGVFDQVNIIMALTMQESGGRSLDIMQSSESIGLPPNSITDPERSIEVGIKHFKKVFKQAGGDVRLTLQAYNFGSGFIDYVKKNGGKYTKKLALDFSRLQAFKMGWKSYGDPSYVDHVMRYVKGSDKNVKPVKGSMDFYETVMKEALKYEGQPYAWGGSNPETGFDCSGLVQWSFAKAGITLPRTAQEQHGATKKISEKEATAGDLVFFGGTYEGKAITHVGIYVGNGRMFNSNDSGIQYSDLKSGYWRDHLVSFGRIK

SEQ ID NO: 60 - DNA, 31 bp, Artificial
  Oligonucleotide as PCR primer BF-YOJL
gcgcggatcctcaaacattcaaataggttcg

SEQ ID NO: 61 - DNA, 29 bp, Artificial
  Oligonucleotide as PCR primer KR-YOJL
gcgcggtaccggtcaatcattgtctggta

SEQ ID NO: 62 - DNA, 28 bp, Artificial
  Oligonucleotide as PCR primer BF-YVCE
gcgcggatccgaaggcgcgatcagcgtt

SEQ ID NO: 63 - DNA, 31 bp, Artificial
  Oligonucleotide as PCR primer KR-YVCE
gcgcggtaccgctcaacacgattaaatgtat

SEQ ID NO: 64 - DNA, 31 bp, Artificial
  Oligonucleotide as PCR primer BF-YDDH
gcgcggatccgatttctatgaaacggtcatg

SEQ ID NO: 65 - DNA, 31 bp, Artificial
  Oligonucleotide as PCR primer KR-YDDH
gcgcggtacccactcctagttatttaatgcg

SEQ ID NO: 66 - DNA, 1242 bp, Bacillus subtilis
  CDS (1)..(1242)
gtgaacacactggcaaactggaagaagtttttgcttgtggcggttatcatttgttttttggttccaattatgacaaaagcggagattgcggaagctgatacatcatcagaattgattgtcagcgaagcaaaaaacctgcttggatatcagtataaatatggcggggaaacgccgaaagagggtttcgatccatcaggattgatacaatatgtgttcagtaaggctgatattcatctgccgagatctgtaaacgaccagtataaaatcggaacagctgtaaaaccggaaaacctgaagccgggtgatattttgtttttcaagaaagagggaagcaccggcactgttccgacacatgacgccctttatatcggagacggccaaatggttcacagtacacagtcaaaaggggttatcatcaccaattacaaaaaaagcagctattggagcggaacttatatcggggcgagacgaatcgctgccgatccggcaacggctgatgttcctgtcgttcaggaggccgaaaaatatatcggtgtcccatatgtgtttggcggaagcacgccgtcagagggctttgattgctcggggcttgtgcaatatgtgtttcaacaggcactcggcatttatctaccgcgatcagccgaacagcagtgggcagtgggcgagaaggtagcccctcagaacataaagcctggtgatgtcgtctatttcagcaatacgtataaaacgggaatttcacatgcaggcatttatgcgggcgcaggcaggttcattcaggcaagccggtcagaaaaagtaaccatttcctatttgtcagaggattactggaaatcgaagatgacgggtattcgccgatttgacaacctgacaatcccgaaagaaaatccgattgtttccgaagcgacgctttatgtcggagaagtgccttacaaacagggcggagtaacacctgagacgggatttgatacagctggatttgtccaatatgtataccagaaagcagccggtatttccctgcctcgatacgcaacaagccagtacaatgccggaactaagattgagaaggcggacctgaagccgggagacattgtgttctttcaatcaacaagcttaaatccctccatctatatcggaaacggacaagttgttcatgtcacattatcaaacggcgtgaccattaccaatatgaacacgagcacatattggaaggataaatacgcaggaagtatacgggtgcaataa

SEQ ID NO: 67 - protein, 413 aa, Bacillus subtilis
VNTLANWKKFLLVAVIICFLVPIMTKAEIAEADTSSELIVSEAKNLLGYQYKYGGETPKEGFDPSGLIQYVFSKADIHLPRSVNDQYKIGTAVKPENLKPGDILFFKKEGSTGTVPTHDALYIGDGQMVHSTQSKGVIITNYKKSSYWSGTYIGARRIAADPATADVPVVQEAEKYIGVPYVFGGSTPSEGFDCSGLVQYVFQQALGIYLPRSAEQQWAVGEKVAPQNIKPGDVVYFSNTYKTGISHAGIYAGAGRFIQASRSEKVTISYLSEDYWKSKMTGIRRFDNLTIPKENPIVSEATLYVGEVPYKQGGVTPETGFDTAGFVQYVYQKAAGISLPRYATSQYNAGTKIEKADLKPGDIVFFQSTSLNPSIYIGNGQVVHVTLSNGVTITNMNTSTYWKDKYAGSIRVQ

SEQ ID NO: 68 - DNA, 31 bp, Artificial
  Oligonucleotide as PCR primer YwtD-FL-Fw
gcgcggtaccgatacatcatcagaattgatt

SEQ ID NO: 69 - DNA, 30 bp, Artificial
  Oligonucleotide as PCR primer YwtD-FL-Rv
gcgcctgcagttattgcacccgtatacttc

(19) United States
(12) Patent Application Publication
(10) Pub. No.: US 2010/0297703 A1
(43) Pub. Date: Nov. 25, 2010

(54) Lytic Enzyme Inhibitor, Lysis Inhibitor, Inhibitor of Poly-y-glutamic Acid Degradation, and Method for Producing Poly- y-glutamic Acid

(76) Inventors: Katsutoshi Ara, Tochigi (JP); Junichi Sekiguchi, Nagano (JP); Hiroki Yamamoto, Nagano (JP); Hiroyuki Harada, Nagano (JP)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/445,667

(22) PCT Filed: Oct. 16, 2007
(86) PCT No.: PCT/JP2007/070513

§ 371 Date: Oct. 16, 2009

(51) Int. Cl. C12P 21/00; C07H 21/04; C07K 14/00; C12N 1/00; C12N 1/20

(52) U.S. Cl. 435/71.2; 536/23.7; 530/350; 435/243; 435/252.5

(57) ABSTRACT

This invention relates to inhibition of microbial lysis via regulation of activity of a lytic enzyme for degrading the cell wall. The lytic enzyme inhibitor and the lysis inhibitor are each composed mainly of the Bacillus subtilis-derived YoeB gene or the YoeB protein encoded by a gene homologous to the YoeB gene. An example of the YoeB protein is a protein consisting of the amino acid sequence as shown in SEQ ID NO: 2.

### TECHNICAL FIELD

The present invention relates to a lytic enzyme inhibitor capable of inhibiting lytic activity of a given Bacillus subtilis-derived lytic enzyme, a lysis inhibitor capable of inhibiting microbial lysis, a microorganism whose lysis is inhibited, an inhibitor of poly-γ-glutamic acid degradation, and a method for producing poly-γ-glutamic acid.

### BACKGROUND ART

Lytic enzymes of Bacillus subtilis play key roles in life cycles, such as cell division, lysis of mother cells in the sporulation phase, germination of spores, and the like. Bacillus subtilis has different lytic enzymes that are specifically expressed in accordance with, for example, the process of cell division or sporulation phase. Many of such lytic enzymes have a binding domain for binding to a cell wall and an active domain to cause a cell wall to lyse. As lytic enzymes that express at the logarithmic growth phase of Bacillus subtilis, CwlB (see Kuroda, A. and J. Sekiguchi, 1991, “Molecular cloning and sequencing of a major Bacillus subtilis autolysin gene,” J. Bacteriol. 173: 7304-7312 and Lazarevic, V., P. Margot, B. Sold, and D. Karamata, 1992, “Sequencing and analysis of the Bacillus subtilis lytRABC divergone: a regulatory unit encompassing the structural gene of the N-acetylmuramoyl-L-alanine amidase and its modifier,” J. Gen. Microbiol. 138: 1949-1961) and CwlG (see Margot, P., C. Mauel, and D. Karamata, 1994, “The gene of the N-acetyl-glucosaminidase, a Bacillus subtilis cell wall hydrase not involved in vegetative cell autolysis,” J. Bacteriol. 12: 535-545 and Rashid, M. H., M. Mori, and J. Sekiguchi, 1995. “Glucosaminidase of Bacillus subtilis: cloning, regulation, primary structure and biochemical characterization,” Microbiology 141: 2391-2404) are known. As lytic enzymes that express at the final stage of cell division of Bacillus subtilis, CwlE (see Margot, P., M. Pagni, and D. Karamata, 1999, “Bacillus subtilis 168 gene lytF encodes a γ-D-glutamate-meso-diaminopimelate muropeptidase expressed by the alternative vegetative sigma factor, σD,” Microbiology 145: 57-65 and Ohnishi, R., S. Ishikawa, and J Sekiguchi, 1999. “Peptidoglycan hydrase LytF plays a role in cell separation with LytE during vegetative growth of Bacillus subtilis,” J. Bacteriol. 181: 3178-3184) and CwlF (see Ishikawa, S., Y. Hara, R. Ohnishi, and J. Sekiguchi, 1998, “Regulation of a new cell wall hydrase gene, CwlF, which affects cell separation in Bacillus subtilis,” J. Bacteriol. 180: 2549-2555 and Margot, P., M. Wahlen, A. Gholamhuseinian, P. Piggot, and D. Karamata, 1998, “The lytE gene of Bacillus subtilis 168 encodes a cell wall hydrase,” J. Bacteriol. 80: 749-752) are known.

Action mechanisms of lytic enzymes on the cell wall of the Bacillus subtilis, however, are not completely understood, and a method for inhibiting activity of the lytic enzymes has not yet been established. If activity of the lytic enzyme can be inhibited, microbial lysis can also be inhibited.

Poly-γ-glutamic acid (hereafter abbreviated to “PGA”) is a polymer compound in which a carboxyl group at position γ is bound to an amino group at position α of glutamic acid via peptide bond. PGA is known as a sticky substance produced by Bacillus subtilis var. natto, and it has drawn attention as a novel polymeric material because of its various properties in recent years. From the viewpoint of various applications as a material, in particular, PGA of a relatively high molecular weight is preferable. However, a technique for producing PGA of a relatively high molecular weight with the use of a microorganism capable of producing PGA has not yet been known.

### DISCLOSURE OF THE INVENTION

Under the above circumstances, the present invention intends to provide a lytic enzyme inhibitor that can regulate activity of a lytic enzyme for degrading a cell wall and a lysis inhibitor that can inhibit microbial lysis. The present invention is also intended to provide a method for producing PGA of a relatively high molecular weight and a method for inhibiting PGA degradation.

In order to attain the above objects, the present inventors have conducted concentrated studies. As a result, they discovered that a given gene product whose functions are unknown would inhibit activity of a lytic enzyme for degrading a cell wall and such gene product would inhibit activity of an enzyme that degrades PGA (i.e., decrease molecular weight). This has led to the completion of the present invention.

The lytic enzyme inhibitor, the lysis inhibitor, and the inhibitor of PGA degradation of the present invention are composed mainly of the Bacillus subtilis-derived YoeB gene or the YoeB protein encoded by a gene homologous to the YoeB gene.

The term “YoeB protein” used herein can be defined as any of proteins (a) to (c) below:

(a) a protein consisting of the amino acid sequence as shown in SEQ ID NO: 2;

(b) a protein consisting of an amino acid sequence derived from the amino acid sequence as shown in SEQ ID NO: 2 by substitution, deletion, addition, or insertion of 1 or several amino acids and having activity of binding to the Bacillus subtilis-derived lytic enzyme to inhibit lytic activity thereof; and

(c) a protein consisting of an amino acid sequence having 70% or higher homology to the amino acid sequence as shown in SEQ ID NO: 2 and having activity of binding to the Bacillus subtilis-derived lytic enzyme to inhibit lytic activity thereof.

The lytic enzyme inhibitor of the present invention can particularly inhibit lysis caused by the CwlE protein, CwlF protein, CwlS protein, CwlO protein, or YddH protein.

The “CwlE protein” can be defined as any of proteins (a) to (c) below:

(a) a protein consisting of the amino acid sequence as shown in SEQ ID NO: 4;

(b) a protein consisting of an amino acid sequence derived from the amino acid sequence as shown in SEQ ID NO: 4 by substitution, deletion, addition, or insertion of one or several amino acids and having activity of lysing the cell wall of Bacillus subtilis; and

(c) a protein consisting of an amino acid sequence having 70% or higher homology to the amino acid sequence as shown in SEQ ID NO: 4 and having activity of lysing the cell wall of Bacillus subtilis.

The “CwlF protein” can be defined as any of proteins (a) to (c) below:

(a) a protein consisting of the amino acid sequence as shown in SEQ ID NO: 6;

(b) a protein consisting of an amino acid sequence derived from the amino acid sequence as shown in SEQ ID NO: 6 by substitution, deletion, addition, or insertion of one or several amino acids and having activity of lysing the cell wall of Bacillus subtilis; and

(c) a protein consisting of an amino acid sequence having 70% or higher homology to the amino acid sequence as shown in SEQ ID NO: 6 and having activity of lysing the cell wall of Bacillus subtilis.

Further, the lysis inhibitor of the present invention can inhibit lysis of Bacillus subtilis, in particular. Bacillus subtilis strains are not limited to wild-type Bacillus subtilis strains, and an example thereof is a mutant lacking at least one type of secretory protease.

The “CwlS protein” can be defined as any of proteins (a) to (c) below:

(a) a protein consisting of the amino acid sequence as shown in SEQ ID NO: 55;

(b) a protein consisting of an amino acid sequence derived from the amino acid sequence as shown in SEQ ID NO: 55 by substitution, deletion, addition, or insertion of one or several amino acids and having activity of lysing the cell wall of Bacillus subtilis; and

(c) a protein consisting of an amino acid sequence having 70% or higher homology to the amino acid sequence as shown in SEQ ID NO: 55 and having activity of lysing the cell wall of Bacillus subtilis.

The “CwlO protein” can be defined as any of proteins (a) to (c) below:

(a) a protein consisting of the amino acid sequence as shown in SEQ ID NO: 57;

(b) a protein consisting of an amino acid sequence derived from the amino acid sequence as shown in SEQ ID NO: 57 by substitution, deletion, addition, or insertion of one or several amino acids and having activity of lysing the cell wall of Bacillus subtilis; and

(c) a protein consisting of an amino acid sequence having 70% or higher homology to the amino acid sequence as shown in SEQ ID NO: 57 and having activity of lysing the cell wall of Bacillus subtilis.

The “YddH protein” can be defined as any of proteins (a) to (c) below:

(a) a protein consisting of the amino acid sequence as shown in SEQ ID NO: 59;

(b) a protein consisting of an amino acid sequence derived from the amino acid sequence as shown in SEQ ID NO: 59 by substitution, deletion, addition, or insertion of one or several amino acids and having activity of lysing the cell wall of Bacillus subtilis; and

(c) a protein consisting of an amino acid sequence having 70% or higher homology to the amino acid sequence as shown in SEQ ID NO: 59 and having activity of lysing the cell wall of Bacillus subtilis.

The inhibitor of PGA degradation of the present invention can inhibit degradation of poly-γ-glutamic acid caused by the CwlE protein, CwlF protein, CwlS protein, or CwlO protein, among the above lytic enzymes. Among the above lytic enzymes, the YddH protein would not exhibit activity of PGA degradation, unlike the CwlE, CwlF, CwlS, and CwlO proteins.

Further, a microorganism of the present invention expresses the Bacillus subtilis-derived YoeB gene or a gene homologous to the YoeB gene downstream of the expression-inducible promoter so that lysis of the microorganism has been inhibited. The microorganism of the present invention particularly preferably lacks at least one type of secretory protease.

Also, the microorganism of the present invention is preferably capable of producing PGA. The microorganism of the present invention can inhibit activity of the lytic enzyme to degrade PGA by the YoeB protein. Examples of lytic enzymes that inhibit PGA degrading activity include the Bacillus subtilis-derived CwlE, CwlF, CwlS, and CwlO proteins and proteins functionally equivalent to the aforementioned proteins.

The method for producing PGA of the present invention comprises culturing microorganisms capable of producing PGA in the presence of the Bacillus subtilis-derived YoeB gene or the YoeB protein encoded by a gene homologous to the YoeB gene and recovering PGA produced in the medium. The method for producing PGA of the present invention may involve the use of microorganisms that express the Bacillus subtilis-derived YoeB gene or a gene homologous to the YoeB gene downstream of the expression-inducible promoter. Alternatively, the method may involve the addition of the YoeB protein to the medium. According to the method for producing PGA of the present invention, the above-mentioned microorganisms produce PGA while activity of PGA degradation of the PGA degrading enzyme is inhibited by the YoeB protein.

This description includes part or all of the contents as disclosed in the description and/or drawings of Japanese Patent Application No. 2006-281768, which is a priority document of the present application.

### BRIEF DESCRIPTION OF THE DRAWINGS

FIG. 1(A) is a photograph showing the results of SDS-PAGE analysis of the outer surface protein purified with the anti-FLAG antibody extracted from the WE1YoeB3FL strain (lane 1) and the outer surface protein purified with the anti-FLAG antibody extracted from the WE1E3FL strain (lane 2). FIG. 1(B) is a photograph showing the results of SDS-PAGE analysis of the outer surface protein purified with the anti-FLAG antibody extracted from the WE1YoeB3FL strain (lane 1) and the results of zymography involving the addition of a cell-wall-component as a substrate (lane 2).

FIG. 2(A) is a photograph showing the results of SDS-PAGE analysis of the outer surface protein purified with the anti-FLAG antibody extracted from the WE1E3FL strain (lane 1) and of the outer surface protein purified with the anti-FLAG antibody extracted from the WE15xLysM3FL strain (lane 2). FIG. 2(B) is a photograph showing the results of SDS-PAGE analysis of the outer surface protein purified with the anti-FLAG antibody extracted from the WE1F3FL strain (lane 1) and of the outer surface protein purified with the anti-FLAG antibody extracted from the WE13xLysM3FL strain (lane 2).

FIG. 3 is a characteristic diagram showing the correlation between the reaction time and decrease in turbidity, when using a cell wall component as a substrate, of a sample to which His-CwlECTD has been added, a sample to which His-CwlECTD and His-YoeB have been added at 1:1, and a sample to which His-CwlECTD and His-YoeB have been added at 1:2.

FIG. 4 is a characteristic diagram showing the correlation between the reaction time and decrease in turbidity, when using a cell wall component as a substrate, of a sample to which the h-ACwlS protein has been added and a sample to which the h-ACwlS protein and the His-YoeB have been added at 1:2.

FIG. 5 is a characteristic diagram showing the correlation between the reaction time and decrease in turbidity, when using a cell wall component as a substrate, of a sample to which the 6His-C-CwlO protein has been added and a sample to which the 6His-C-CwlO protein and the His-YoeB have been added at 1:2.

FIG. 6 is a characteristic diagram showing the correlation between the reaction time and decrease in turbidity, when using a cell wall component as a substrate, of a sample to which the 6His-C-YddH protein has been added and a sample to which the 6His-C-YddH protein and His-YoeB have been added at 1:2.

FIG. 7 is a characteristic diagram showing the effects of lysis inhibition in the secretory protease-deficient Dpr8 strain, when YoeB proteins are overexpressed.

FIG. 8 is a photograph showing the results of SDS-polyacrylamide electrophoresis representing the results of comparison and examination of activity of the CwlE protein for PGA degradation in the presence of and in the absence of the YoeB protein.

FIG. 9 is a photograph showing the results of SDS-polyacrylamide electrophoresis representing the results of comparison and examination of activity of the CwlF protein for PGA degradation in the presence of and in the absence of the YoeB protein.

FIG. 10 is a photograph showing the results of SDS-polyacrylamide electrophoresis representing the results of comparison and examination of activity of the CwlS protein for PGA degradation in the presence of and in the absence of the YoeB protein.

FIG. 11 is a photograph showing the results of SDS-polyacrylamide electrophoresis representing the results of comparison and examination of activity of the CwlO protein for PGA degradation in the presence of and in the absence of the YoeB protein.

FIG. 12 is a photograph showing the results of SDS-polyacrylamide electrophoresis representing the results of comparison and examination of activity of the YwtD protein for PGA degradation in the presence of and in the absence of the YoeB protein.

### BEST MODES FOR CARRYING OUT THE INVENTION

Hereafter, the present invention is described in detail with reference to the drawings.

The lytic enzyme inhibitor, the lysis inhibitor, and the inhibitor of PGA degradation of the present invention are composed mainly of the Bacillus subtilis YoeB gene or the YoeB protein encoded by a gene homologous to the YoeB gene.

An example of the nucleotide sequence of the Bacillus subtilis YoeB gene is shown in SEQ ID NO: 1, and the amino acid sequence of the YoeB protein encoded by the nucleotide sequence as shown in SEQ ID NO: 1 is shown in SEQ ID NO: 2. The YoeB protein encoded by the Bacillus subtilis YoeB gene is not limited to a polypeptide consisting of the amino acid sequence as shown in SEQ ID NO: 2. It may be a polypeptide consisting of an amino acid sequence derived from the amino acid sequence as shown in SEQ ID NO: 2 by substitution, deletion, addition, or insertion of one or a plurality of amino acids, which has activity of inhibiting the lytic activity of the lytic enzyme (hereafter referred to as a “mutant polypeptide”). The number of amino acids to be substituted, deleted, added, or inserted can be, for example, 1 to 20, preferably 1 to 10, and more preferably 1 to 5. A region in which one or a plurality of amino acids are substituted, deleted, added, or inserted is, for example, a region excluding a region between positions 24 and 181 of the amino acid sequence as shown in SEQ ID NO: 2. The region between positions 24 and 181 is considered to bind to a lytic enzyme. Accordingly, activity of inhibiting lytic activity of the lytic enzyme can be retained, even if one or a plurality of amino acids are substituted, deleted, added, or inserted in a region excluding the above region.

The YoeB protein encoded by the Bacillus subtilis YoeB gene is not limited to a polypeptide consisting of an amino acid sequence as shown in SEQ ID NO: 2. It may be a polypeptide having 70% or higher, preferably 80% or higher, more preferably 90% or higher, and most preferably 95% or higher homology to the amino acid sequence as shown in SEQ ID NO: 2, which has activity of binding to the Bacillus subtilis-derived lytic enzyme to inhibit lytic activity thereof (hereafter, it is also referred to as a “mutant polypeptide”). A homology value is a value determined by default using software that computes homology among a plurality of amino acid sequences (e.g., DANASYS or BLAST).

The Bacillus subtilis YoeB gene is not limited to a polynucleotide consisting of the nucleotide sequence as shown in SEQ ID NO: 1. It may be a polynucleotide that hybridizes under stringent conditions to a complementary strand of the polynucleotide consisting of the nucleotide sequence as shown in SEQ ID NO: 1, which encodes a polypeptide having activity of inhibiting lytic activity of the lytic enzyme (hereafter, it is referred to as a “mutant polynucleotide”). A mutant polynucleotide encodes a polypeptide consisting of an amino acid sequence different from the amino acid sequence as shown in SEQ ID NO: 2, which has activity of inhibiting lytic activity of the lytic enzyme (hereafter, it is referred to as a “mutant polypeptide”).

Stringent conditions refer to conditions that so-called specific hybrids are formed, but non-specific hybrids are not formed. Under stringent conditions, for example, highly homologous DNAs hybridize to each other (e.g., homology therebetween is 50% or higher), and DNAs less homologous to each other would not hybridize. More specifically, as a stringent condition, hybridization takes place at 60° C. and a salt concentration equivalent to 1×SSC, 0.1% SDS, and preferably 0.1×SSC, 0.1% SDS, which are washing conditions for general Southern hybridization. A mutant polynucleotide can be obtained by introducing a given mutation into a wild type YoeB gene derived from Bacillus subtilis. A method for introducing mutation is not particularly limited, and so-called site-directed mutagenesis can be employed.

Whether or not the various above-mentioned mutant polypeptides have the activity of inhibiting the lytic activity of the lytic enzyme can be determined in the following manner. Specifically, a mutant containing a gene encoding a mutant polypeptide substituted with a wild-type YoeB gene is prepared. Alternatively, a mutant where a wild-type YoeB gene is disrupted and a mutant polypeptide is introduced is prepared. Such mutant is cultured and morphologically observed. When a mutant polypeptide has activity of inhibiting lytic activity of the lytic enzyme, the shape of the mutant is rounder than that of a wild-type strain. When a mutant polypeptide does not have activity of inhibiting lytic activity of the lytic enzyme, the shape of the mutant becomes rod-like, as with a wild-type strain. Thus, morphological observation of the mutant expressing a gene that encodes a mutant polypeptide enables determination of whether or not the mutant polypeptide has the activity of inhibiting lytic activity of the lytic enzyme.

The main component of the lytic enzyme inhibitor, the lysis inhibitor, or the inhibitor of PGA degradation of the present invention is not limited to a protein encoded by the Bacillus subtilis-derived YoeB gene. The main component may be the YoeB protein encoded by a homologous gene corresponding to the Bacillus subtilis YoeB gene, which has been isolated from a microorganism other than Bacillus subtilis. An example of a microorganism other than Bacillus subtilis is Bacillus licheniformis.

Hereafter, a method for isolating a homologous gene of the YoeB gene from a microorganism other than Bacillus subtilis is described. However, a person skilled in the art can isolate a target homologous gene in accordance with a conventional technique, without being limited to the technique described below.

At the outset, a database storing nucleotide sequence information is used to identify a highly homologous gene using the nucleotide sequence as shown in SEQ ID NO: 1 as the query sequence. Subsequently, primers for specifically amplifying the identified gene are designed and chemically synthesized. Subsequently, genome DNA is extracted from the target microorganism in accordance with a conventional technique. The target homologous gene of the YoeB gene can be amplified via PCR using the extracted genome DNA as a template and the above primers, and then be isolated.

Whether or not the isolated homologous gene of the YoeB gene has functions equivalent to those of the YoeB protein can be examined by preparing an expression vector into which the isolated homologous gene of the YoeB gene has been incorporated, transforming the same into the target microorganism, and observing the shape when the transformed microorganism is cultured under conditions in which the above homologous gene is expressed. When the isolated homologous gene of the YoeB gene has functions equivalent to those of the YoeB protein, the shape of the transformed microorganism becomes rounder than that of the wild-type strain. When the isolated homologous gene of the YoeB gene has functions similar to those of the YoeB protein, the shape of the transformed microorganism would be similar to that of the wild-type strain.

The Bacillus subtilis YoeB gene and a gene homologous to the YoeB gene can be used to prepare the YoeB protein, which is a main component of the lytic enzyme inhibitor and the lysis inhibitor of the present invention. The YoeB protein may be produced with the use of the above Bacillus subtilis YoeB gene or a gene homologous to the YoeB gene by using a recombinant vector, which is prepared by incorporating the gene of interest into a vector that can replicate and maintain the gene of interest in a host cell, can stably express the enzyme, and can stably maintain the gene, thereby transforming the host microorganism.

When an E. coli host is used, a vector is not particularly limited, and pUC18, pBR322, pHY300PLK (Yakult Honsha Co., Ltd.), and other vectors can be used. When a Bacillus subtilis host is used, pUB110, pHSP64 (Sumitomo et al., Biosci. Biotechnol, Biocem., 59, 2172-2175, 1995), pHY300PLK, or other vectors can be used, without particular limitation.

Host cells can be transformed via a protoplast method, a competent cell method, electroporation, or other techniques. A host cell is not particularly limited, and examples include Gram-positive strains such as Bacillus subtilis, Gram-negative strains such as E. coli, Actinomycetes such as Streptomyces, yeast such as Saccharomyces, and fungi such as Aspergillus.

The resulting transformants may be cultured under adequate conditions using a medium containing carbon sources, nitrogen sources, metal salts, vitamins, and the like assimilable by microorganisms. The resulting culture solution may be subjected to enzyme fractionation or purification in accordance with conventional techniques to obtain YoeB proteins having activity of inhibiting lytic activity of the lytic enzyme via concentration via ultrafiltration, lyophilization, spray drying, crystallization, or the like.

When the lytic enzyme inhibitor and the lysis inhibitor of the present invention are added to a medium in which microorganisms are cultured or to a buffer in which microorganisms are suspended, activity of lytic enzymes expressed by the microorganisms can be lowered, and lysis of such microorganisms can be inhibited. Lytic enzymes that are inhibited by the lytic enzyme inhibitor of the present invention are not particularly limited, and examples thereof include a lytic enzyme that is expressed in a cell-cycle-dependent manner and a lytic enzyme that is expressed in a cell-cycle-independent manner. In particular, the lytic enzyme inhibitor of the present invention can inhibit lysis caused by the CwlE, CwlF, CwlS, CwlO, and YddH proteins, which are lytic enzymes of Bacillus subtilis. The CwlE, CwlF, CwlS, CwlO, and YddH proteins are known to be lytic enzymes that are expressed at the cell-division surface and at both poles of the cell at the final stage of Bacillus subtilis cell division. The CwlE, CwlF, CwlS, CwlO, and YddH proteins each contain a signal peptide, a cell-wall-binding domain, and a active domain for cell wall lysis (i.e., a D,L-endopeptidase domain) in that order from the N-terminus. The lytic enzyme inhibitor and the lysis inhibitor of the present invention bind to the active domain for cell wall lysis of the CwlE, CwlF, CwlS, CwlO, and YddH proteins and inhibit cell wall lytic activity of the CwlE, CwlF, CwlS, CwlO, and YddH proteins to inhibit lysis.

When the inhibitor of PGA degradation of the present invention is added at the time of culture of microorganisms capable of producing PGA, PGA degrading activity of lytic enzymes having activity of PGA degradation can be lowered, and decrease of molecular weights of PGA produced by the microorganisms can be prevented. Examples of lytic enzymes to be inhibited by the inhibitor of PGA degradation of the present invention include CwlE, CwlF, CwlS, and CwlO proteins. PGA with a relatively high molecular weight can be produced by lowering PGA degrading activity of lytic enzymes.

The nucleotide sequence of the gene encoding the CwlE protein (the CwlE gene) is shown in SEQ ID NO: 3, and the amino acid sequence of the CwlE protein is shown in SEQ ID NO: 4. The nucleotide sequence of the gene encoding the CwlF protein (the CwlF gene) is shown in SEQ ID NO: 5, and the amino acid sequence of the CwlF protein is shown in SEQ ID NO: 6. The nucleotide sequence of the gene encoding the CwlS protein (the CwlS gene) is shown in SEQ ID NO: 54, and the amino acid sequence of the CwlS protein is shown in SEQ ID NO: 55. The nucleotide sequence of the gene encoding the CwlO protein (the CwlO gene) is shown in SEQ ID NO: 56, and the amino acid sequence of the CwlO protein is shown in SEQ ID NO: 57. The nucleotide sequence of the gene encoding the YddH protein (the YddH gene) is shown in SEQ ID NO: 58, and the amino acid sequence of the YddH protein is shown in SEQ ID NO: 59.

The term “protein that is functionally equivalent to the Bacillus subtilis-derived CwlE, CwlF, CwlS, CwlO, or YddH protein” refers to, for example, a protein consisting of an amino acid sequence derived from the amino acid sequence as shown in SEQ ID NO: 4, 6, 55, 57, or 59 by substitution, deletion, addition, or insertion of 1 or several amino acids and having activity of lysing the cell wall of Bacillus subtilis or a protein consisting of an amino acid sequence having 70% or higher, 80% or higher, 90% or higher, more preferably 95% or higher, further preferably 96% or higher, particularly preferably 97% or higher, more particularly preferably 98% or higher, and most preferably 99% or higher homology to the amino acid sequence as shown in SEQ ID NO: 4, 6, 55, 57, or 59 and having activity of lysing the cell wall of Bacillus subtilis. The lytic enzyme inhibitor of the present invention can act on such proteins.

The number of amino acids to be substituted, deleted, added, or inserted can be, for example, 1 to 40, preferably 1 to 20, and more preferably 1 to 10. A region in which one or a plurality of amino acids are substituted, deleted, added, or inserted is, for example, a region excluding a region between positions 371 and 488 of the amino acid sequence as shown in SEQ ID NO: 4, a region between positions 218 and 334 of the amino acid sequence as shown in SEQ ID NO: 6, a region between positions 1 and 290 of the amino acid sequence as shown in SEQ ID NO: 55, a region between positions 1 and 343 of the amino acid sequence as shown in SEQ ID NO: 57, and a region between positions 1 and 206 of the amino acid sequence as shown in SEQ ID NO: 59. The region between positions 371 and 488 of the amino acid sequence as shown in SEQ ID NO: 4, the region between positions 218 and 334 of the amino acid sequence as shown in SEQ ID NO: 6, the region between positions 1 and 290 of the amino acid sequence as shown in SEQ ID NO: 55, the region between positions 1 and 343 of the amino acid sequence as shown in SEQ ID NO: 57, and the region between positions 1 and 260 of the amino acid sequence as shown in SEQ ID NO: 59 are considered to be regions to which the lytic enzyme inhibitor of the present invention binds. Accordingly, activity of the lytic enzyme inhibitor of the present invention can be retained, even if one or a plurality of amino acids are substituted, deleted, added, or inserted in a region excluding the above regions.

As described above, the lytic enzyme inhibitor and the lysis inhibitor of the present invention enable inhibition of cell wall lysing activity of the lytic enzyme via binding of the YoeB protein to the active domain of the lytic enzyme for cell wall lysis, such as the CwlE, CwlF, CwlS, CwlO, or YddH protein. This inhibits lysis of the microorganisms that express the above lytic enzymes and enables culture for a relatively long period of time. When a material is produced with the use of a microorganism such as Bacillus subtilis, for example, the amount of material production can be increased if each cell can be cultured for a longer period of time. With the addition of the lytic enzyme inhibitor of the present invention when culturing microorganisms that produce materials of interest, the efficiency of production of such materials can be improved.

The lysis inhibitor of the present invention can be applied to a wide range of microorganisms. Application thereof to bacteria of Bacillus, such as Bacillus subtilis, is particularly preferable. The target microorganisms of the lysis inhibitor of the present invention are not limited to wild-type microorganisms. For example, mutants having a variety of mutations can also be targets. An example of a target microorganism of the lysis inhibitor of the present invention is a mutant that lacks at least one type of secretory protease. Since such mutant lacks secretory protease, it is preferable as a host that produces a material, such as a useful protein, to the outside of the cell. A system that produces the material of interest with the use of such mutant can inhibit the lysis of the mutant through the addition of the lysis inhibitor of the present invention. This enables culture of the mutant for a relatively long period of time. Consequently, the efficiency of the production of such materials can be improved, and the cost of material production can be reduced.

The amount of the lytic enzyme inhibitor or the lysis inhibitor of the present invention to be added is not particularly limited. Such amount can adequately be determined, provided that such inhibitors can bind to the active domain of the lytic enzyme for cell wall lysis, such as the CwlE, CwlF, CwlS, CwlO, or YddH protein.

As described above, the inhibitor of PGA degradation of the present invention enables inhibition of PGA degrading activity of the lytic enzyme via binding of the YoeB protein to the active domain of the lytic enzyme having activity of PGA degradation for cell wall lysis, such as the CwlE, CwlF, CwlS, or CwlO protein. When microorganisms having the capacity for PGA production and expressing the lytic enzyme are cultured in the presence of the inhibitor of PGA degradation, accordingly, PGA of a relatively high molecular weight can be produced. The term “PGA of a relatively high molecular weight” refers to PGA having a molecular weight that is significantly higher than the average molecular weight of PGA produced by the microorganisms in the absence of the inhibitor of PGA degradation of the present invention. Since the inhibitor of PGA degradation of the present invention can inhibit microbial lysis as described above, PGA of a relatively high molecular weight can be produced for a long period of time.

The inhibitor of PGA degradation of the present invention can be applied to a wide range of microorganisms capable of PGA production. Examples of microorganisms capable of PGA production include Bacillus subtilis var. natto, Bacillus licheniformis, Bacillus megaterium, Bacillus anthracis, Bacillus halodurans, and Natrialba aegyptiaca, Hydra.

The present invention can provide microorganisms that are modified so as to express the lytic enzyme inhibitor or the gene encoding the lysis inhibitor under the control of an expression-inducible promoter. By culturing the microorganism of the present invention under the expression-inducible conditions, the lysis inhibitor is secreted to the medium, which in turn enables inhibition of the lysis for a relatively long period of time.

Accordingly, the microorganism of the present invention having such constitution may be utilized to produce materials, such as useful proteins, to enhance the efficiency of the production of such materials. The microorganism of the present invention is particularly preferably modified to express the lytic enzyme inhibitor or the gene encoding the lysis inhibitor downstream of the expression-inducible promoter with the use of a mutant that lacks at least one type of secretory protease as a host. Such constitution enables prevention of degradation of target substances, such as useful proteins, which in turn allows realization of higher production efficiency.

The term “expression-inducible promoter” used herein refers to a promoter that has a function of enhancing expression of the gene located downstream in the presence of a given material. An expression-inducible promoter used for the microorganism of the present invention is not particularly limited. An example is a promoter (P_{spac}) that is regulated by a lac operon repressor, i.e., lacl, which induces expression of downstream genes in the presence of IPTG.

As the microorganism of the present invention, the microorganism capable of PGA production is particularly preferably modified so as to express the gene encoding the inhibitor of PGA degradation under the control of the expression-inducible promoter. Culture of such microorganisms in a medium enables production of PGA of a relatively high molecular weight. When such microorganisms are cultured in a medium to produce PGA of a high molecular weight, separately prepared inhibitor of PGA degradation may be added to the medium.

Hereafter, the present invention is described in greater detail with reference to the examples, although the technical scope of the present invention is not limited thereto.

### Example 1

### Examination of YoeB Protein-Lytic Enzyme Interaction

In this example, the interaction between the YoeB protein (SEQ ID NO: 2) encoded by the YoeB gene (SEQ ID NO: 1) and the CwlE protein encoded by the CwlE gene (SEQ ID NO: 3) was examined.

Specifically, the WE1YoeB3FL strain that expresses the YoeB-3×FLAG protein induced with IPTG and the WE1E3FL strain that expresses the CwlE-3×FLAG protein induced with IPTG were first constructed. The WE1YoeB3FL strain and the WE1E3FL strain were prepared using the WE1 strain (Yamamoto, H. et al., 2003 “Localization of the vegetative cell wall hydrases LytC, LytE, LytF on the Bacillus subtilis cell surface and stability of these enzymes to cell wall-bound or extracellular proteases,” J. Becteriol., 185: 6666-6677). The WE1 strain has a genotype in which the minor extracellular serine protease (i.e., the epr gene) is disrupted via insertion of a tetracycline-resistant gene and the cell wall associate protease (i.e., the wprA gene) is disrupted via insertion of a kanamycin-resistant gene.

The WE1YoeB3FL strain was prepared by transforming the pCA3FLYoeB vector containing the gene encoding the YoeB-3×FLAG protein downstream of the IPTG-inducible promoter into the WE1 strain. The WE1E3FL strain was prepared by transforming the pCA3FLCE vector containing the gene encoding the CwlE-3×FLAG protein downstream of the IPTG-inducible promoter into the WE1 strain. A 3×FLAG-fused protein can be detected using the anti-FLAG M2 monoclonal antibody (Sigma).

Subsequently, the outer surface protein was extracted from the constructed WE1YoeB3FL and WE1E3FL strains, respectively. Specifically, these strains were each inoculated in LB agar medium and subjected to pre-pre-culture at 37° C. for 12 hours. The resulting single colony was inoculated in 5 ml of LB liquid medium and then subjected to pre-culture at 30° C. for 10 hours. After the pre-culture, the pre-cultured strain was inoculated in 50 ml of LB liquid medium to bring the OD₆₀₀ value to 0.01, and main culture was conducted until the OD₆₀₀ value reached 2.0. After the completion of the culture, the culture solution was centrifuged at 10,000 rpm for 10 minutes. The supernatant was removed and the precipitate was washed with 25 mM Tris-HCl (pH 7.2). The washed precipitate was suspended in 3M LiCl (25 mM Tris-HCl, pH 7.2) to a density of 10 μl/OD, and the suspension was allowed to stand on ice for 20 minutes. Thereafter, centrifugation was carried out at 4° C. and 10,000 rpm for 10 minutes. The supernatant was recovered, TCA (trichloroacetic acid) was added to a final concentration of 5%, the mixture was thoroughly mixed, and the resulting suspension was allowed to stand on ice for 60 minutes. Thereafter, centrifugation was carried out at 4° C. and 15,000 rpm for 5 minutes. The supernatant was removed, and the precipitate was rinsed with 70% ethanol to thoroughly remove TCA. Thus, outer surface proteins were extracted from the WE1YoeB3FL strain and the WE1E3FL strain, respectively.

Subsequently, 200 μl of affinity gel (Anti-FLAG M2 Affinity Gel Freezer-Safe (Sigma)) substituted with TBS buffer (50 mM Tris-HCl, 150 mM NaCl (pH 7.4)) was added and mixed. The mixture was mildly (8 rpm) agitated at 4° C. for 1 hour. Thereafter, centrifugation was carried out at 5,000 rpm for 1 minute. The supernatant was removed while carefully refraining from suctioning the precipitated gel. Thereafter, 500 μl of TBS buffer was added to prepare a suspension. Thereafter, the gel was transferred to the Spin Column (Micro Bio Spin Chromatography Columns (BIO RAD)), and the gel was trapped via centrifugation at 1,000 g for 30 seconds. Thereafter, the resultant was washed three times with 500 μl of TBS buffer. Subsequently, 100 μl of Glycine HCl elution buffer (0.1M glycine-HCl) was sequentially added thereto and thoroughly agitated. Elution was carried out 1 minute later at 1,000 g for 30 seconds, and this procedure was repeated two times. The products were combined to prepare an eluted solution.

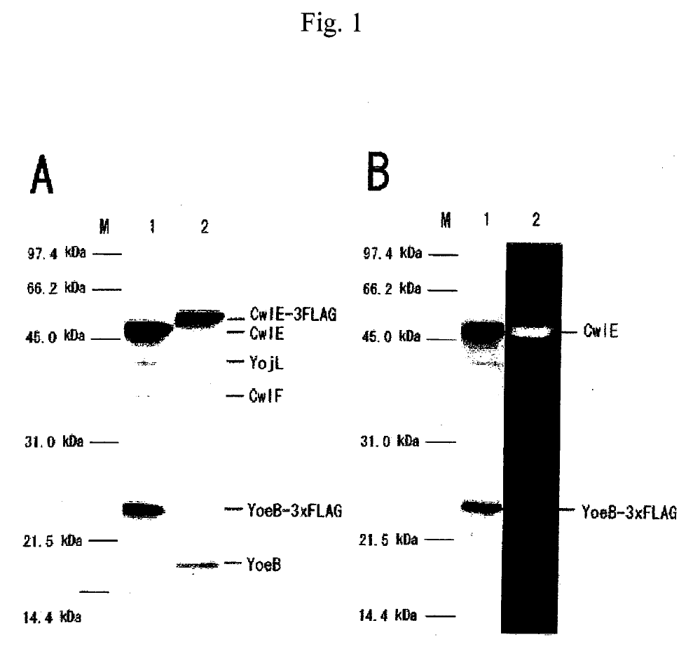

Subsequently, the band pattern of the resulting eluted solution was inspected via SDS-PAGE. Electrophoresis was carried out by charging proteins in amounts corresponding to the amount extracted from the cells (OD₆₀₀=200) to the relevant lanes. The results are shown in FIG. 1A. In FIG. 1A, lane 1 represents an outer surface protein purified with the anti-FLAG antibody extracted from the WE1YoeB3FL strain and lane 2 represents an outer surface protein purified with the anti-FLAG antibody extracted from the WE1E3FL strain.

As is apparent from lane 1 of FIG. 1A, a band was detected at positions corresponding to the CwlE protein, the CwlF protein, and the YojL protein in the outer surface protein purified with the anti-FLAG antibody extracted from the WE1YoeB3FL strain. The integrated values and the proportions thereof are shown in Table 1.

TABLE 1

| | | | Integrated | |
|---|---|---|---|---|
| | Integrated | Molecular | value/Molecular | Proportion relative |
| | value | weight (Da) | weight | to YoeB (1) |
| CwlE | 30057 | 51229 | 0.59 | 0.89 |
| YojL | 989 | 44073 | 0.02 | 0.03 |
| CwlF | 1247 | 37162 | 0.03 | 0.05 |
| YoeB | 13201 | 19984 | 0.66 | 1.00 |

The results demonstrated that the YoeB protein were bound to the CwlE, CwlF, and YojL proteins when OD₆₀₀ of the cells was 2.0. The YojL protein is a component known as peptidoglycan hydrolase (the DL-endopeptidase II family). As is apparent from lane 2 of FIG. 1A, a band was detected at the position corresponding to the YoeB protein in the outer surface protein purified with the anti-FLAG antibody extracted from the WE1E3FL strain.

In order to verify that a protein interacting with the YoeB protein was the CwlE protein, zymography was carried out using a cell wall component as a substrate. The cell wall component was prepared in the following manner. Specifically, Bacillus subtilis 168 strain was first cultured in a 4-liter system, harvested, suspended in a 4M LiCl solution, and then boiled for 15 minutes. The solution was cooled to room temperature, glass beads (φ0.1 mm) were added thereto, and the strains were fragmented using a homogenizer for 1 hour, followed by fragmentation using ultrasound waves for 30 minutes. The resulting solution of fragmented strains was allowed to stand and the supernatant was recovered. The supernatant was recovered after centrifugation at 3,000 rpm for 5 minutes to completely remove glass beads. Subsequently, centrifugation was carried out at 13,000 rpm for 10 minutes to recover the cell wall component in the precipitated fraction. The precipitated fraction was then suspended in a 4% (w/v) SDS solution, and the resultant was boiled for 15 minutes. Then, the resultant was cooled to room temperature and then washed with ion-exchange water until SDS was completely removed. The resulting solution was designated as a solution of cell wall component. The density of the cell wall component in the solution was calculated to be 1.1 mg/ml based on the assayed OD₅₄₀ value of 1.0.

Zymography was carried out in the following manner. At the outset, the cell wall component was added to a separation gel to a final density of 0.05% when performing SDS-PAGE. After the completion of electrophoresis, the gel was mildly shaken in Renaturation Buffer (25 mM Tris-HCl (pH 7.2), 1% Triton-X100) at 37° C. for 1 to 16 hours. Thereafter, the resultant was soaked in a staining solution (0.01% methylene blue, 0.01% KOH), mildly shaken at room temperature, and then decolored with ion-exchange water until an active band became visually observable.

The results are shown in FIG. 1B. In FIG. 1B, lane 1 represents an outer surface protein purified with the anti-FLAG antibody extracted from the WE1YoeB3FL strain and lane 2 represents the results of zymography carried out with the use of a cell wall component as a substrate. As shown in lane 2 of FIG. 1B, a band of substantially the same intensity was detected at the same position as in the case of the CwlE protein as a protein that had interacted with the YoeB protein.

The results shown in FIGS. 1A and 1B demonstrated that the YoeB protein would bind to the CwlE protein.

Subsequently, the domain of the CwlE protein to which the YoeB protein had bound was inspected. The CwlE protein comprises a signal peptide, a cell-wall-binding domain, and an active domain in that order from the N-terminus. Thus, the WE15xLysM3FL strain expressing a mutant CwlE-3×FLAG protein having no active domain and containing a signal peptide, a cell-wall-binding domain, and a FLAG tag in that order from the N-terminus induced with IPTG, was constructed.

The WE1E3FL strain and the WE15xLysM3FL strain mentioned above were used, the outer surface proteins were purified using the anti-FLAG antibody, and the band pattern was inspected via SDS-PAGE in the same manner as in the above experiment. Electrophoresis of the WE1E3FL strain was carried out by charging proteins in amounts corresponding to the amount extracted from the cells (OD₆₀₀=200) to the relevant lanes. Electrophoresis of the WE15xLysM3FL strain was carried out by charging proteins in amounts corresponding to the amount extracted from the cells (OD₆₀₀=2,000) to the relevant lanes.

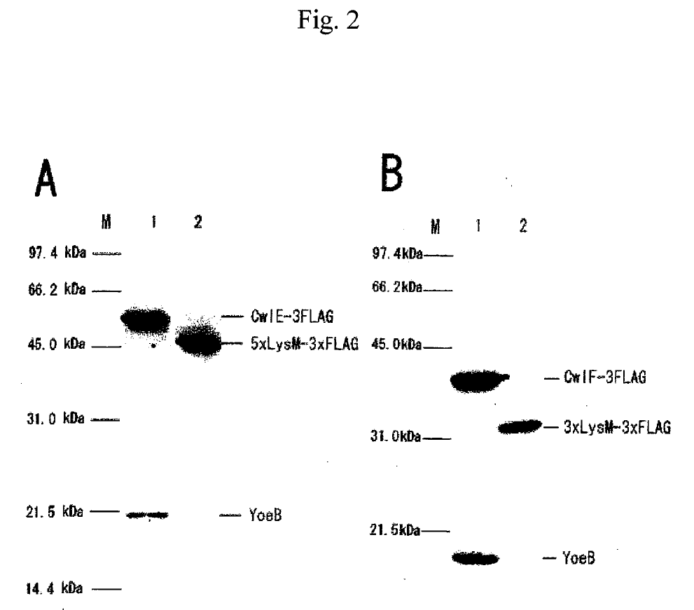

The results are shown in FIG. 2A. In FIG. 2A, lane 1 represents an outer surface protein purified with the anti-FLAG antibody extracted from the WE1E3FL strain and lane 2 represents an outer surface protein purified with the anti-FLAG antibody extracted from the WE15xLysM3FL strain. As is apparent from FIG. 2A, a band of the YoeB protein was detected in lane 1 but was not detected in lane 2. Such results demonstrated that the YoeB protein was bound to an active domain of the CwlE protein.

As shown in FIG. 1A, the YoeB protein was also found to have been bound to the CwlF protein. Thus, the site of the CwlF protein interacting with the YoeB protein was also inspected. The CwlF protein is a major cell wall degrading enzyme of Bacillus subtilis, and it is known to exhibit a localization pattern similar to that of the CwlE protein (i.e., a cell division surface and both poles).

In this experiment, the WE1F3FL strain that expresses the CwlF-3×FLAG protein induced with IPTG and the WE13xLysM3FL strain that expresses the mutant CwlF-3×FLAG protein having no an active domain and containing a signal peptide, a cell wall binding domain, and a FLAG tag in that order from the N-terminus induced with IPTG were constructed. The WE1F3FL strain and the WE13xLysM3FL strain mentioned above were used, the outer surface proteins were purified using the anti-FLAG antibody, and the band pattern was inspected via SDS-PAGE in the same manner as in the above experiment.

The results are shown in FIG. 2B. In FIG. 2B, lane 1 represents an outer surface protein purified with the anti-FLAG antibody extracted from the WE1F3FL strain and lane 2 represents an outer surface protein purified with the anti-FLAG antibody extracted from the WE13xLysM3FL strain. As is apparent from FIG. 2B, a band of the YoeB protein was detected in lane 1 but was not detected in lane 2. Such results demonstrated that the YoeB protein was also bound to an active domain of the CwlF protein as in the case of the CwlE protein.

### Example 2

### Effects of YoeB Protein for Inhibiting Lytic Enzyme Activity 1

In this example, the way that the YoeB protein would act on the cell wall lysing activity of a lytic enzyme was inspected via activity assay in liquid. In this example, cell wall lysing activity of the C-terminal active domain (CwlECTD) of CwlE was assayed using the cell wall component prepared in the same manner as in Example 1 as a substrate in the presence of or in the absence of the YoeB protein.

In this example, cell wall lysing activity was assayed using 20 mM MES buffer (pH 6.5). The cell wall component in an amount corresponding to OD₅₄₀=0.6 was washed with the assay buffer, and the cell wall was precipitated via centrifugation. The supernatant was removed, 2 ml of assay buffer was added to the precipitate to prepare a suspension, a cell wall component was prepared to adjust the final OD₅₄₀ value to 0.3, and the resultant was designated as a substrate solution.

Activity assay was carried out by allowing the His-tag-fused C-terminal active domain of CwlE (His-CwlECTD) to act on a substrate solution and assaying the turbidity of the substrate solution. Specifically, the cell wall lysing activity of His-CwlECTD was evaluated in terms of decrease in turbidity. The turbidity of the substrate solution was assayed using the ultraviolet-visible spectrophotometer (V-560, JASCO Corporation). Decrease in turbidity was determined by designating the OD₅₄₀ value 0 minutes after the initiation of assay as 100% and calculating the decrease based on the OD₅₄₀ values 5, 10, 20, and 30 minutes thereafter.

In this example, a sample prepared by adding 2 nmol (29 μg) of His-CwlECTD to a substrate solution, a sample prepared by adding 2 nmol (29 μg) of His-CwlECTD and 2 nmol (38.1 μg) of His-YoeB to a substrate solution, and a sample prepared by adding 2 nmol (29 μg) of His-CwlECTD and 4 nmol (76.1 μg) of His-YoeB to a substrate solution were assayed.

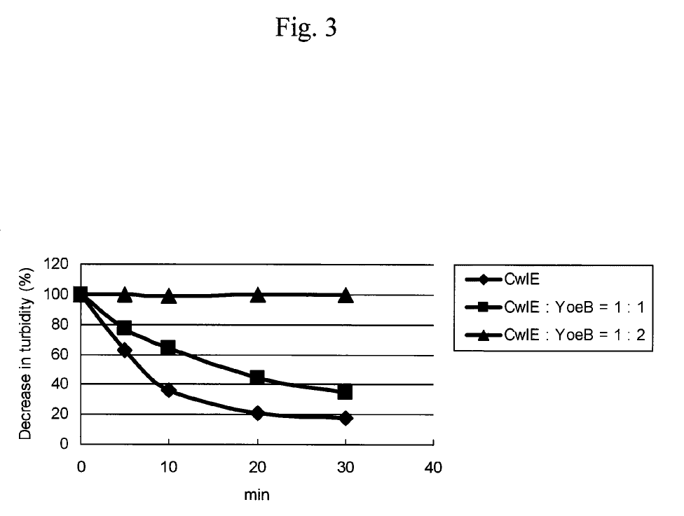

The correlation between the reaction time and decrease in turbidity is shown in FIG. 3. As is apparent from FIG. 3, the OD₅₄₀ value of the sample prepared by adding His-CwlECTD to the substrate solution began to significantly decrease immediately after the initiation of assay. Thus, His-CwlECTD was found to have strong lytic activity on the cell wall. In contrast, the sample prepared by adding the equivalent amount of His-CwlECTD and His-YoeB by mole to the substrate solution exhibited a decrease in the OD₅₄₀ value immediately after the initiation of assay; however, a milder curve was obtained than with a sample prepared by adding His-CwlECTD only. This indicates that activity of His-CwlECTD could be inhibited with the addition of the equivalent amount of His-YoeB by mole. In the case of a sample prepared by adding His-CwlECTD and His-YoeB at a ratio of 1:2 by mole, the OD₅₄₀ value did not substantially decrease immediately after the initiation of assay, and the value remained at a substantially constant level. This indicates that addition of His-YoeB in an amount twice the amount of His-CwlECTD would inhibit substantially 100% activity of His-CwlECTD by mole.

### Example 3

### Effects of YoeB Protein for Inhibiting Lytic Enzyme Activity 2

In this example, cell wall lysing activity of the C-terminal active domains of CwlS, CwlO, and YddH was assayed using the cell wall component prepared in the same manner as in Example 1 as a substrate in the presence of or in the absence of the YoeB protein.

### <Preparation of C-Terminal Active Domain of CwlS>

The 3′-terminal DNA sequence of the cwlS gene corresponding to the D,L-endopeptidase domain (including a region from nucleotide 290 to the termination codon and a 61-bp transcription terminator-like sequence) was amplified via PCR using the BF-YOJL primer (gcgcggatcctcaaacattcaaataggttcg: SEQ ID NO: 60) and the KR-YOJL primer (gcgcggtaccggtcaatcattgtctggta: SEQ ID NO: 61) and the genomic DNA extracted from the Bacillus subtilis 168 strain as a template. The underlined nucleotide sequences of the above BF-YOJL and KR-YOJL primers indicate the BamHI and KpnI restriction enzyme recognition sequences, respectively. After amplification via PCR, the resulting PCR product was digested with the BamHI and KpnI restriction enzymes to obtain an insert.

Subsequently, the pQE30 plasmid was digested with BamHI and KpnI, and the resultant was ligated to the above insert to construct the pQEΔojL plasmid. The pQE30 plasmid was developed for the purpose of expressing an His-tag fusion protein in E. coli.

Whether or not a DNA sequence of the insert had been properly inserted was verified via sequencing. The pQEΔojL plasmid, which was verified to have been properly inserted, was introduced into the E. coli JM109 strain by the competent method. The E. coli M13(pREP4) strain into which the pQEΔojL plasmid had been inserted is hereafter referred to as “M13 (pQEΔojL).”

M13 (pQEΔojL) was cultured in 200 ml of LB medium containing 100 μg/ml ampicillin, 25 μg/ml kanamycin, and 2% glucose at 37° C. When OD₆₀₀ of the culture solution was 1.0, IPTG (isopropyl 1-thio-β-D-galactoside) was added to the medium to a final concentration of 1 mM, and culture was conducted for an additional 5 hours. After culture, the culture product was centrifuged (8,000 rpm, 10 minutes, 4° C.) to harvest cells, and the cells were suspended in the initiation buffer (5 ml; 1× phosphate buffer (pH 7.0) containing 10 mM imidazole).

Subsequently, the resultant was subjected to ultrasonic fragmentation (on: 0.5 seconds; off: 1 second/on: 30 seconds; off: 30 seconds; 10 minutes in total) and then centrifugation (12,000 rpm, 10 minutes, 4° C.). The resulting supernatant was filtered through a 0.45-mm filter. The h-ACwlS protein extracted via filtration was purified in accordance with a conventional technique of His-Trap column purification of GE Healthcare and Biosciences. The purified h-ACwlS protein solution was further dialyzed with 20 mM phosphate buffer (pH 7.0) to prepare the h-ACwlS protein solution.

### <Preparation of C-Terminal Active Domain of CwlO>

The 3′-terminal DNA sequence of the cwlO gene corresponding to the D,L-endopeptidase domain (including a region from nucleotide 343 to the termination codon and an 84-bp transcription terminator-like sequence) was amplified via PCR using the BF-YVCE primer (gcgcggatccgaaggcgcgatcagcgtt: SEQ ID NO: 62) and the KR-YVCE primer (gcgcggtaccgctcaacacgattaaatgtat: SEQ ID NO: 63) and the genomic DNA extracted from the Bacillus subtilis 168 strain as a template. The underlined nucleotide sequences of the above BF-YVCE and KR-YVCE primers indicate the BamHI and KpnI restriction enzyme recognition sequences, respectively.

The resulting PCR product was used to prepare the pQEVCE plasmid in the same manner as with the above <Preparation of C-terminal active domain of CwlS>, and the E. coli M13 (pQEVCE) into which pQEVCE had been introduced was obtained. The obtained M13 (pQEVCE) was used to prepare the 6His-C-CwlO protein solution in the same manner as with the above <Preparation of C-terminal active domain of CwlS>.

### <Preparation of C-Terminal Active Domain of YddH>

The 3′-terminal DNA sequence of the YddH gene corresponding to the D,L-endopeptidase domain (including a region from nucleotide 206 to the termination codon and a 9-bp untranslated region) was amplified via PCR using the BF-YDDH primer gcgcggatccgatttctatgaaacggtcatg: SEQ ID NO: 64) and the KR-YDDH primer (gcgcggtacccactcctagttatttaatgcg: SEQ ID NO: 65) and the genomic DNA extracted from the Bacillus subtilis 168 strain as a template. The underlined nucleotide sequences of the above BF-YDDH and KR-YDDH primers indicate the BamHI and KpnI restriction enzyme recognition sequences, respectively.

The resulting PCR product was used to prepare the pQEDDH plasmid in the same manner as with the above <Preparation of C-terminal active domain of CwlS>, and the E. coli M13 (pQEDDH) into which pQEDDH had been introduced was obtained. The obtained M13 (pQEDDH) was used to prepare the 6His-C-YddH protein solution in the same manner as with the above <Preparation of C-terminal active domain of CwlS>.

### <Assay of Cell Wall Lysing Activity and Results Thereof>

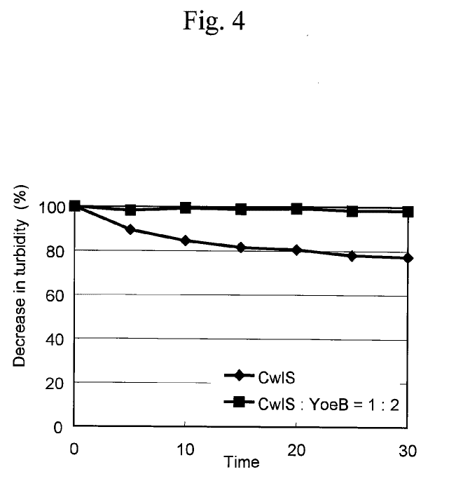

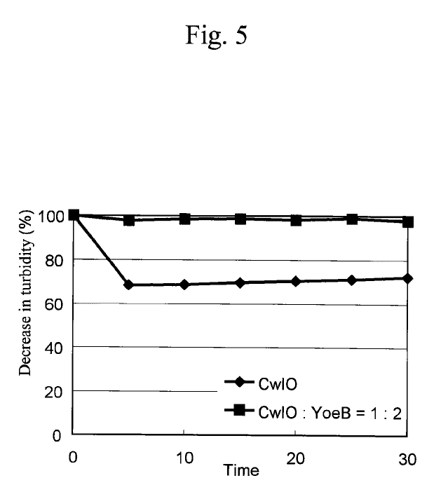

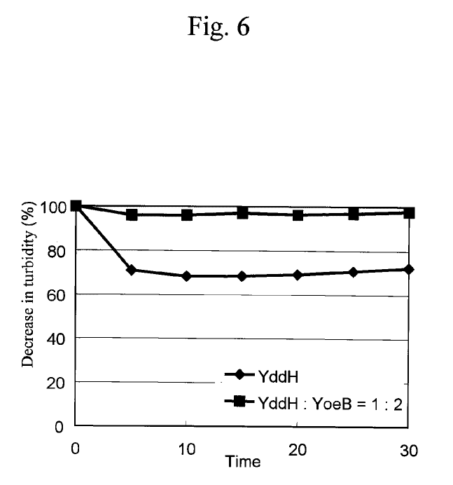

Cell wall degrading activity was assayed in accordance with the method described in Example 2. In this example, the His-YoeB protein was added in twice the amount of the h-ACwlS, 6His-C-CwlO, or 6His-C-YddH protein by mole. The results of assay of the cell wall lysing activity of the CwlS protein are shown in FIG. 4, the results of assay of the cell wall lysing activity of the CwlO protein are shown in FIG. 5, and the results of assay of the cell wall lysing activity of the YddH protein are shown in FIG. 6.

As shown in FIGS. 4 to 6, the YoeB protein was found to be capable of inhibiting the cell wall lysing activity of lytic enzymes, such as CwlS, CwlO, and YddH proteins. By allowing the YoeB protein to act in twice the amount of such enzyme by mole, substantially 100% of the cell wall lysing activity of lytic enzymes was found to be inhibited.

### Example 4

### Effects of YoeB Protein for Inhibiting Lytic Enzyme Activity 3

In this example, effects of lysis inhibition when YoeB proteins were overexpressed in the Bacillus subtilis mutant Dpr8 lacking 8 types of secretory proteases (Δepr/ΔwprA/Δmpr/ΔnprB/Δbpr/ΔnprE/Δvpr/ΔaprE) were examined. The Dpr8 strain lacks 8 types of known secretory proteases and thus is more likely than a wild-type strain to undergo lysis.

The Dpr8 strain was constructed in the following manner. A list of primers used for constructing the Dpr8 strain is shown in Table 2.

TABLE 2

| | | SEQ ID |
|---|---|---|
| Primer | Nucleotide sequence | NO: |
| eprfw1 | CTCCGGCAGAAAGGGCAT | 7 |
| eprUPr | CGTTCAGCCGTACAACAAGTTTGCAAGACATG | 8 |
| eprDNf | AACTTGTTGTACGGCTGAACGCCGTCAAACC | 9 |
| eprrv-repU | CTGATCTCGACTTCGTTCAGACGGGTCGTACA | 10 |
| | ATGGCTG | |
| repUfw | GAACGAAGTCGAGATCAG | 11 |
| Cmrv1 | GCTGTAATATAAAAACCTTCT | 12 |
| eprfw2 | CGACACCATAGCTTTCTG | 13 |
| Cmrv2 | AACTAACGGGGCAGGTTA | 14 |
| repUr-CmU | AGAATCAATCCCATGGTCTCACTTTTCCACTT | 15 |
| | TTTGTCTTG | |
| CmUf-repU | GTGAGACCATGGGATTGATTCTAATGAAGAAA | 16 |
| | GCAGACAAG | |
| wprAfw1 | GGGTAATTTATCTGATAGGG | 17 |
| wprAUPr | CTTTTGCTTCCCACAACCGAGCTGAATTTTCTG | 18 |
| wprADNf | CTCGGTTGTGGGAAGCAAAAGTTGTTGTTGAAA | 19 |
| | A | |
| wprArv- | CTGATCTCGACTTCGTTCATCCTCATTGAAGAC | 20 |
| repU | GGCATC | |
| wprAfw2 | GGAACATATATGACACACCT | 21 |
| mprfw1 | TGTTTGGTGTTGAGCTGTT | 22 |
| mprUPr | TTCGTGTGAATCCATTGTTTTCTGAATCTTGGA | 23 |
| | A | |
| mprDNf | GAAAACAATGGATTCACACGAACGGAGGATCGT | 24 |
| mprrv-repU | CTGATCTCGACTTCGTTCCTCGTAAGAAAAAAT | 25 |
| | ACCTATTTC | |
| mprfw2 | CCTTGCGAAAGATAGGGTA | 26 |
| nprBfw1 | TTTTTAGCAGTGGTGCTC | 27 |
| nprBUPr | CATACGCCTTCAGTAATAGAGATGTCTTGGTC | 28 |
| nprBDNf | CTCTATTACTGAAGGCGTATGAGGCTGTCGGC | 29 |
| nprBrv-repU | CTGATCTCGACTTCGTTCTTCCAAATGCGCTT | 30 |
| | CATTAGGA | |
| nprBfw2 | CTTTTGAGCCCGTTCCTC | 31 |
| bprfw1 | TGAGATTGTGGTGACAGTG | 32 |
| bprUPr | TTAAGTTTTCCGCTGATGAGTCTGTTTTTCGT | 33 |
| | T | |
| bprDNf | GACTCATCAGCGGAAAACTTAATATGAACACA | 34 |
| | GAA | |
| bprrv-repU | CTGATCTCGACTTCGTTCGTAATCATGACACC | 35 |
| | GTTTTGAAC | |
| bprfw2 | ATTGCAACCGGCTTTATCG | 36 |
| nprEfw1 | TTTTGAAGACGTTCGGCGA | 37 |
| nprEUPr | ATTGTCTGTTGCTGAAGCAGCCTGGAATGCTG | 38 |
| | TT | |
| nprEDNf | CAGGCTGCTTCAGCAACAGACAATTTCTTACC | 39 |
| | TA | |
| nprErv-repU | CTGATCTCGACTTCGTTCCCTCTCTTAAGTAA | 40 |
| | GCGCTG | |
| nprEfw2 | TCTCTTTGTTGAAAAACGATA | 41 |
| vprfw1 | AAAAACATCCCTCCGCTTC | 42 |
| vprUPr | TCTTCGGTCAATACAAGCAGAAAGCGAATGAT | 43 |
| vprDNf | TCTGCTTGTATTGACCGAAGAACCTTTCACTG | 44 |
| vprrv-repU | CTGATCTCGACTTCGTTCTGCTCGGCTCATCT | 45 |
| | GGAGAAA | |
| eprfw2 | TTTTTGGCAGGCAGCCTT | 46 |
| aprEfw1 | CTGTTTATTATGGGCCACGAA | 47 |
| aprEUPr | GATCAGCTTGGGGTTAATCAACGTACAAGCAG | 48 |
| aprEDNf | TGATTAACCCCAAGCTGATCCACAATTTTTTG | 49 |
| | C | |
| aprErv-repu | CTGATCTCGACTTCGTTCTGATTTTCCAAACG | 50 |
| | AGCTTTC | |
| aprEfw2 | ATGGGCCATTATGTCATGAAG | 51 |

Primers used for the deletion of the genes are summarized in Table 3.

TABLE 3

| For deletion | For deletion | For deletion | For deletion | For deletion | For deletion | For deletion | For deletion |
|---|---|---|---|---|---|---|---|
| of epr gene | of wprA gene | of mpr gene | of nprB gene | of bpr gene | of nprE gene | of vpr gene | of aprE gene |
| eprfw1 | wprAfw1 | mprfw1 | nprBfw1 | bprfw1 | nprEfw1 | vprfw1 | aprEfw1 |
| eprUPr | wprAUPr | mprUPr | nprBUPr | bprUPr | nprEUPr | vprUPr | aprEUPr |
| eprDNf | wprADNf | mprDNf | nprBDNf | bprDNf | nprEDNf | vprDNf | aprEDNf |
| eprrv-repU | wprArv-repU | mprrv-repU | nprBrv-repU | bprrv-repU | nprErv-repU | vprrv-repU | aprErv-repu |
| eprfw2 | wprAfw2 | mprfw2 | nprBfw2 | bprfw2 | nprEfw2 | vprfw2 | aprEfw2 |
| Cmrv2 | Cmrv2 | Cmrv2 | Cmrv2 | Cmrv2 | Cmrv2 | Cmrv2 | Cmrv2 |
| repUfw | repUfw | repUfw | repUfw | repUfw | repUfw | repUfw | repUfw |
| Cmrv1 | Cmrv1 | Cmrv1 | Cmrv1 | Cmrv1 | Cmrv1 | Cmrv1 | Cmrv1 |
| repUr-CmU | repUr-CmU | repUr-CmU | repUr-CmU | repUr-CmU | repUr-CmU | repUr-CmU | repUr-CmU |
| CmUf-repU | CmUf-repU | CmUf-repU | CmUf-repU | CmUf-repU | CmUf-repU | CmUf-repU | CmUf-repU |

A method for deleting the epr gene from the wild-type Bacillus subtilis 168 strain is first described. Genomic DNA extracted from the Bacillus subtilis 168 strain was used as a template, a primer set of eprfwl and eprUpr and a primer set of eprDNf and eprrv-rep shown in Table 2 were used, and a 0.6-kb fragment (A) adjacent to a region upstream of the epr gene and a 0.5-kb fragment (B) adjacent to a region downstream of the epr gene of the genome were prepared. Separately, a promoter region of the repU gene (Nucleic Acids Res., 17, 4410, 1989) derived from the pUB110 plasmid (Plasmid 15, 93, 1986) was ligated to a upstream region of the chloramphenicol resistance gene derived from the pC194 plasmid (J. Bacteriol. 150 (2), 815, 1982) to prepare a 1.2-kb fragment (C). Subsequently, the 3 obtained fragments (A), (B), and (C) were mixed to prepare a template, and SOE-PCR was carried out using the eprfw2 and Cmrv2 primers shown in Table 2 to ligate the 3 fragments in the order of (A), (B), and (C) to obtain a 2.2-kb DNA fragment. The terminuses of the DNA fragment were blunt-ended and 5′-phosphorylated, and the resultant was inserted into the SmaI restriction enzyme site of the pUC118 plasmid (Methods Enzymol. 153, 3, 1987) to construct the pUC118-CmrΔepr plasmid for deleting the epr gene. The aforementioned 1.2-kb fragment (C) was prepared via SOE-PCR using a mixture of a 0.4-kb fragment (D) and a 0.8-kb fragment (E) as a template and the repUfw and Cmrvl primers shown in Table 2. Specifically, the 0.4-kb fragment (D) contains the repU gene promoter region prepared with the use of the primer set of repUfw and repUr-Cm (Table 2) and the pUB110 plasmid as a template, and the 0.8-kb fragment (E) contains the chloramphenicol resistance gene prepared with the use of the primer set of CmUf-rep and Cmrvl and the pC194 plasmid as a template.

Subsequently, the pUC118-CmrΔepr plasmid for deleting the epr gene prepared as described above was introduced into the Bacillus subtilis 168 strain by the competent cell transformation technique (J. Bacteriol. 93, 1925, 1967), and a transformed strain fused with genomic DNA via single-cross-over homologous recombination between corresponding upstream or downstream regions of the epr gene was obtained using chloramphenicol resistance as an indicator. The resulting transformed strain was inoculated in LB medium, cultured at 37° C. for 2 hours, and then subjected to competence induction again. Thus, homologous intergenomic recombination was induced between the overlapping upstream or downstream regions of the genome of the epr gene. When homologous intergenomic recombination takes place in a region different from the region at the time of plasmid introduction, the epr gene would be deleted along with dropout of the plasmid-derived chloramphenicol resistance gene and the pUC118 vector region. Subsequently, ampicillin concentration was carried out in the following manner, in order to enhance the abundance of chloramphenicol-sensitive strains. The culture solution after the competent cells were induced was inoculated in 1 ml of LB medium containing chloramphenicol (final concentration: 5 ppm) and ampicillin sodium (final concentration: 100 ppm) to the turbidity at 600 nm (OD₆₀₀) of 0.003. After culture was conducted at 37° C. for 5 hours, 10 μl of an aqueous solution of 10,000 ppm ampicillin sodium was added, and culture was conducted for an additional 3 hours. After the completion of culture, strains were washed via centrifugation with the use of an aqueous solution of 2% sodium chloride, the resultant was suspended in 1 ml of an aqueous solution of 2% sodium chloride, and LB agar medium was coated with 100 μl of the suspension. The strains were incubated at 37° C. for approximately 15 hours, and the strains that had become sensitive to chloramphenicol because of dropout of the plasmid region were selected from among the grown strains. The genomic DNA of the selected strains was used as a template to perform PCR using the eprfw2 and eprrv-rep primers shown in Table 2, thereby confirming deletion of the epr gene. Thus, the epr gene-deficient strain was obtained.

Subsequently, the resulting epr gene-deficient strain was subjected to the next deletion; i.e., deletion of the wprA gene, in the same manner as with the deletion of the epr gene. Specifically, the pUC118-CmrΔwprA plasmid for deletion of the wprA gene was constructed in the same manner, the resulting plasmid was introduced into genomic DNA, and the wprA gene was deleted via subsequent homologous intergenomic recombination to obtain a strain lacking both the epr and wprA genes. Subsequently, the same procedure was repeated to successively delete mpr, nprB, bpr, nprE, vpr, and aprE genes, a 8-protease-deficient strain lacking 8 types of protease genes was constructed at last, and the strain was designated as the Dpr8 strain.

The pDG148YB plasmid for overexpressing yoeB to be introduced into the Dpr8 strain was prepared in the following manner. At the outset, the yoeBSal-F-2 (5′-GCGCAGATCTGTCGACTAAGAGAAAGAGATTTTGAAGG (SEQ ID NO: 52)) and yoeBSph-R (5′-GCGCGCATGCTTATATAACTGCGTCAAATTG (SEQ ID NO: 53)) primers for amplifying the yoeB gene were used to perform PCR amplification using the chromosome DNA of the wild-type Bacillus subtilis strain as a template. The amplified fragment (595 bp) was digested with SalI and SphI restriction enzymes. The fragment was ligated to the pUC118 plasmid, which had also been digested with SalI and SphI restriction enzymes, to obtain pU8YB. After the nucleotide sequence of the insert was confirmed, pU8YB was digested with SalI and SphI restriction enzymes. The cleaved yoeB gene fragment (575 bp) was ligated to pDG148, which had also been digested with SalI and SphI restriction enzymes, to obtain pDG148YB of interest. pDG148 is a multicopy plasmid having a promoter that is induced to express by IPTG and it is available from the Bacillus Genetic Stock Center.

The pDG148YB prepared as above was introduced into the Dpr8 strain, and the following experiment was carried out. Pre-culture was conducted in LB medium at 30° C. for 15 hours. A pre-culture solution (600 μl) was added to 30 ml of 2×L medium containing 7.5% maltose, 7.5 μg/ml MnSO₄, and 10 μg/ml kanamycin, and main culture was then initiated. In order to induce overexpression of yoeB, IPTG was added to the main culture solution 0, 8, and 20 hours after the initiation of culture to a final concentration of 1 mM in each case. A sample to which IPTG was not added was designated as a control sample. The turbidity of the culture solution at OD₆₀₀ was assayed with the elapse of time, and the effects of lysis inhibition were examined.

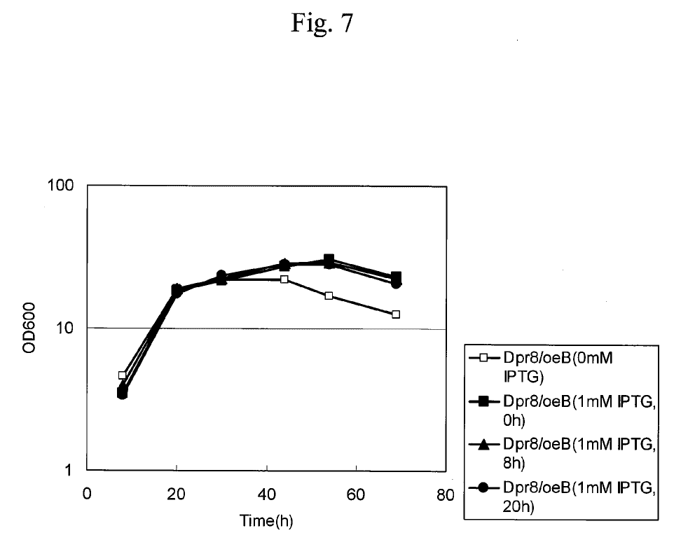

The results are shown in FIG. 7. As is apparent from FIG. 7, the sample to which IPTG was not added began to exhibit lowered turbidity about 30 hours after the initiation of culture. That is, lysis of the Dpr8 strain was observed. In the samples in which YoeB proteins had been overexpressed with the addition of IPTG, however, lowered turbidity was not observed 30 hours after the initiation of culture. This demonstrates that lysis was inhibited in the Dpr8 strain in which YoeB proteins had been overexpressed.

### Example 5

### Activity of YoeB Protein for Inhibiting PGA Degradation

In this example, the effects of the YoeB protein (SEQ ID NO: 2) encoded by the YoeB gene (SEQ ID NO: 1) for inhibiting PGA degradation were examined. Specifically, various types of lytic enzymes were allowed to act on PGAs of a given range of molecular weights, and degradation of PGAs (i.e., reduction of molecular weight) was compared in the presence of or in the absence of the YoeB protein. The various types of lytic enzymes and the YoeB protein used in this example were those used in Examples 2 and 3.

In this example, PGA products (No. 168-21413: Wako Pure Chemical Industries, Ltd.) having average molecular weights of 4,000 kDa to 6,000 kDa were used. The degradation reaction of PGA was carried out using 1 nmol of lytic enzyme and 2 mg of PGA in 10 mM sodium phosphate buffer (pH 7.0). When the YoeB protein was to be added, the amount thereof was 2 nmol. The reaction temperature was 37° C. After a given reaction time had elapsed, the reaction was terminated via boiling at 100° C. for 5 minutes. After the termination of the reaction, the sample was subjected to electrophoresis on 14% SDS-polyacrylamide gel, and PGA degradation was confirmed via methylene blue staining.

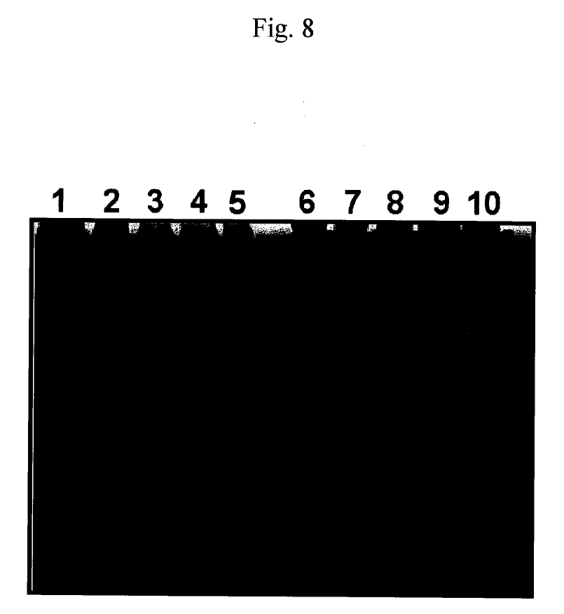

Specifically, the reaction solution having the composition shown in Table 4 was prepared for the CwlE protein. The results of examining the PGA degrading activity of the CwlE protein are shown in FIG. 8.

TABLE 4

Sample No.

| | 1 | 2 | 3 |
|---|---|---|---|
| PGA | 100 μl (2 mg) | 100 μl (2 mg) | 100 μl (2 mg) |
| His-CwlE | — | 23 μl (1 nmol) | 23 μl (1 nmol) |
| His-YoeB | — | — | 96 μl (2 nmol) |
| Buffer | 300 μl | 277 μl | 181 μl |
| Total | 400 μl | 400 μl | 400 μl |

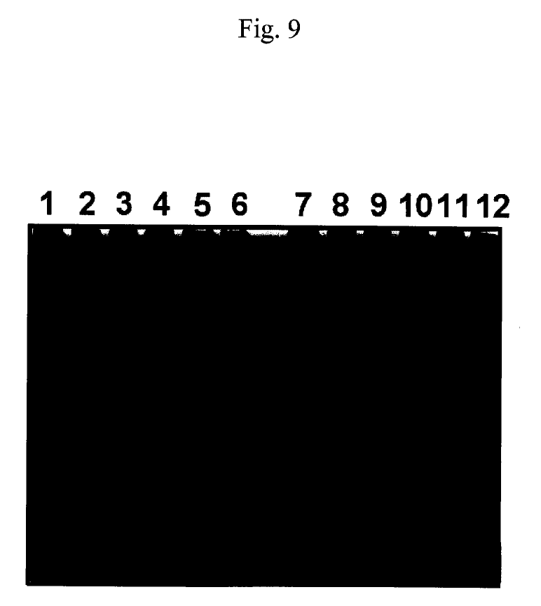

The reaction solution having the composition shown in Table 5 was prepared for the CwlF protein. The results of examining the PGA degrading activity of the CwlF protein are shown in FIG. 9.

TABLE 5

Sample No.

| | 1 | 2 | 3 |
|---|---|---|---|
| PGA | 100 μl (2 mg) | 100 μl (2 mg) | 100 μl (2 mg) |
| His-CwlF | — | 63 μl (1 nmol) | 63 μl (1 nmol) |
| His-YoeB | — | — | 146 μl (2 nmol) |
| Buffer | 300 μl | 237 μl | 91 μl |
| Total | 400 μl | 400 μl | 400 μl |

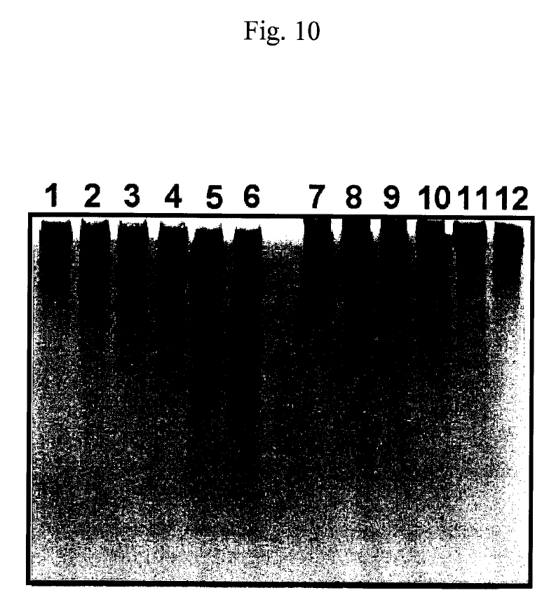

The reaction solution having the composition shown in Table 6 was prepared for the CwlS protein. The results of examining the PGA degrading activity of the CwlS protein are shown in FIG. 10.

TABLE 6

Sample No.

| | 1 | 2 | 3 |
|---|---|---|---|
| PGA | 100 μl (2 mg) | 100 μl (2 mg) | 100 μl (2 mg) |
| His-CwlS | — | 40 μl (1 nmol) | 40 μl (1 nmol) |
| His-YoeB | — | — | 146 μl (2 nmol) |
| Buffer | 300 μl | 260 μl | 114 μl |
| Total | 400 μl | 400 μl | 400 μl |

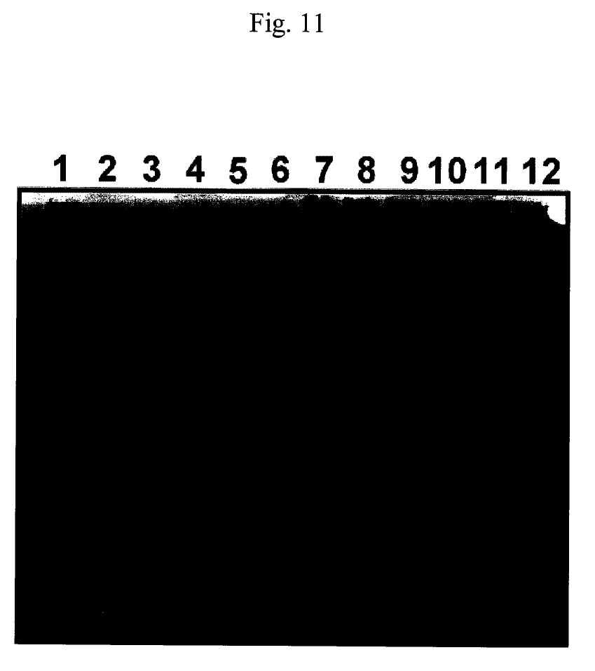

The reaction solution having the composition shown in Table 7 was prepared for the CwlO protein. The results of examining the PGA degrading activity of the Cw10 protein are shown in FIG. 11.

TABLE 7

Sample No.

| | 1 | 2 | 3 |
|---|---|---|---|
| PGA | 100 μl (2 mg) | 100 μl (2 mg) | 100 μl (2 mg) |
| His-CwlO | — | 13 μl (1 nmol) | 13 μl (1 nmol) |
| His-YoeB | — | — | 146 μl (2 nmol) |
| Buffer | 300 μl | 287 μl | 141 μl |
| Total | 400 μl | 400 μl | 400 μl |

In FIG. 9, lane 1 represents a reaction solution of sample No. 1, lanes 2 to 5 represent the reaction solution of sample No. 2 that has been subjected to the reaction for 0.5 hours, 1.0 hour, 2.0 hours, and 4.0 hours, respectively, and lanes 6 to 10 represent the reaction solution of sample No. 3 that has been subjected to the reaction for 0 hours, 0.5 hours, 1.0 hour, 2.0 hours, and 4.0 hours, respectively. In FIGS. 10 and 11, lane 1 represents a reaction solution of sample No. 1, lanes 2 to 6 represent the reaction solution of sample No. 2 that has been subjected to the reaction for 0.5 hours, 1.0 hour, 2.0 hours, 4.0 hours, and 6.0 hours, respectively, and lanes 7 to 12 represent the reaction solution of sample No. 3 that has been subjected to the reaction for 0 hours, 0.5 hours, 1.0 hour, 2.0 hours, 4.0 hours, and 6.0 hours, respectively.

As is apparent from FIGS. 9 to 11, the CwlE, CwlF, CwlS, and CwlO proteins have activity of completely degrading PGA and reducing the molecular weight thereof in the absence of the YoeB protein. Also, activity of PGA degradation of the CwlE, CwlF, CwlS, and CwlO proteins was found to be inhibited in the presence of the YoeB protein, and PGA was found to maintain the given range of molecular weights without being degraded. This example demonstrated that the YoeB protein has a novel effect of inhibiting PGA degrading activity of the lytic enzyme.

For the purpose of comparison, whether or not the YwtD protein, which is known to have no cell wall lysing activity but to have PGA degrading activity, would exhibit the effects of inhibiting PGA degradation by the YoeB protein was examined. The nucleotide sequence of the YwtD gene is shown in SEQ ID NO: 66, and the amino acid sequence of the YwtD protein is shown in SEQ ID NO: 67.

The YwtD protein was obtained in the same manner as in the case of <Preparation of C-terminal active domain of CwlS> of Example 3. Specifically, the DNA sequence of the YwtD gene containing the D,L-endopeptidase domain was amplified via PCR using the YwtD-FL-Fw primer (gcgcggtaccgatacatcatcagaattgatt: SEQ ID NO: 68) and the YwtD-FL-Rv primer (gcgcctgcagttattgcacccgtatacttc: SEQ ID NO: 69) and the genomic DNA extracted from the Bacillus subtilis 168 strain as a template. The underlined nucleotide sequences of the above YwtD-FL-Fw and YwtD-FL-Rv primers indicate the KpnI and PstI restriction enzyme recognition sequences, respectively.

The resulting PCR product was used to prepare the pQEYwtD plasmid in the same manner as with the above <Preparation of C-terminal active domain of CwlS>, and E. coli M13 (pREP4, pQEYwtD) into which pQEYwtD had been introduced was obtained. The resulting M13 (pREP4, pQEYwtD) was used to prepare the h-YwtD protein solution in the same manner as with the above <Preparation of C-terminal active domain of CwlS>. The PGA degradation reaction using the resulting YwtD protein was carried out by preparing a reaction solution having the composition shown in Table 8 in the same manner as described above.

TABLE 8

Sample No.

| | 1 | 2 | 3 |
|---|---|---|---|
| PGA | 100 μl (2 mg) | 100 μl (2 mg) | 100 μl (2 mg) |
| His-YwtD | — | 77 μl (1 nmol) | 77 μl (1 nmol) |
| His-YoeB | — | — | 96 μl (2 nmol) |
| Buffer | 300 μl | 223 μl | 127 μl |
| Total | 400 μl | 400 μl | 400 μl |

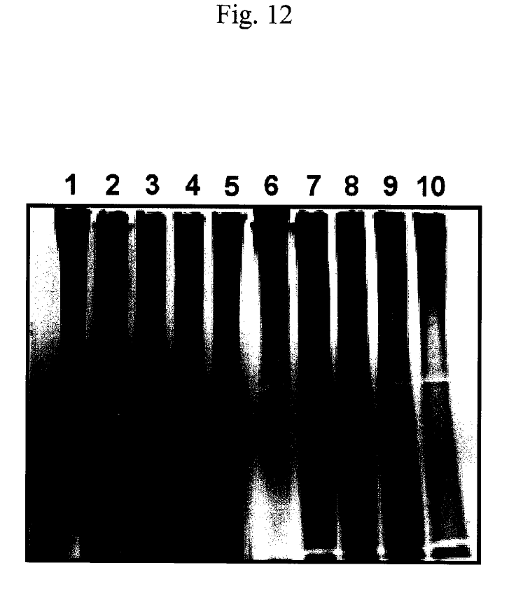

The results are shown in FIG. 12. In FIG. 12, lane 1 represents a reaction solution of sample No. 1, lanes 2 to 5 represent the reaction solution of sample No. 2 that has been subjected to the reaction for 0.5 hours, 1.0 hour, 2.0 hours, and 4.0 hours, respectively, and lanes 6 to 10 represent the reaction solution of sample No. 3 that has been subjected to the reaction for 0 hours, 0.5 hours, 1.0 hour, 2.0 hours, and 4.0 hours, respectively. As is apparent from FIG. 12, PGA degrading activity of the YwtD protein was not different in the presence of and in the absence of the YoeB protein. Specifically, the YoeB protein was found not to inhibit PGA degrading activity of the YwtD protein.

It was thus demonstrated that the YoeB protein would inhibit PGA degrading activity of a lytic enzyme having cell wall lysing activity but would not inhibit PGA degrading activity of a PGA degrading enzyme having no cell wall lysing activity.

### INDUSTRIAL APPLICABILITY

The present invention can provide a novel lytic enzyme inhibitor that can effectively inhibit, for example, activity of a Bacillus subtilis-derived lytic enzyme and a lysis inhibitor that can inhibit, for example, lysis of Bacillus subtilis. Specifically, the lytic enzyme inhibitor of the present invention can inhibit lytic activity of various types of lytic enzymes, including Bacillus subtilis-derived lytic enzymes. Also, the lysis inhibitor of the present invention can inhibit lysis of various types of microorganisms, including Bacillus subtilis.

Also, the present invention can provide a microorganism exhibiting inhibition of lysis by a lytic enzyme. With the use of the microorganism of the present invention as, for example, a host for material production, the productivity of such material can be improved because lysis thereof has been inhibited.

Further, the present invention can provide an inhibitor of PGA degradation that inhibits degradation of PGA caused by a lytic enzyme. Also, the present invention can provide a method for producing a PGA of a higher molecular weight than a wild-type strain.

All publications, patents, and patent applications cited herein are incorporated herein by reference in their entirety.

1. A lytic enzyme inhibitor composed mainly of the Bacillus subtilis-derived YoeB gene or the YoeB protein encoded by a gene homologous to the YoeB gene.

2. The lytic enzyme inhibitor according to claim 1, wherein the YoeB protein is any of proteins (a) to (c) below:
(a) a protein consisting of the amino acid sequence as shown in SEQ ID NO: 2;
(b) a protein consisting of an amino acid sequence derived from the amino acid sequence as shown in SEQ ID NO: 2 by substitution, deletion, addition, or insertion of one or several amino acids and having activity of binding to the Bacillus subtilis-derived lytic enzyme to inhibit lytic activity thereof; and
(c) a protein consisting of an amino acid sequence having 70% or higher homology to the amino acid sequence as shown in SEQ ID NO: 2 and having activity of binding to the Bacillus subtilis-derived lytic enzyme to inhibit lytic activity thereof.

3. The lytic enzyme inhibitor according to claim 1 or 2, which inhibits lytic activity of at least 1 type of lytic enzyme selected from the group consisting of the Bacillus subtilis-derived CwlE, CwlF, CwlS, CwlO, and YddH proteins and proteins functionally equivalent thereto.

4. The lytic enzyme inhibitor according to claim 3, wherein the CwlE protein is any of the proteins (a) to (c) below:
(a) a protein consisting of the amino acid sequence as shown in SEQ ID NO: 4;
(b) a protein consisting of an amino acid sequence derived from the amino acid sequence as shown in SEQ ID NO: 4 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 4 and having activity of lysing the cell wall of Bacillus subtilis.

5. The lytic enzyme inhibitor according to claim 3, wherein the CwlF protein is any of proteins (a) to (c) below:
(a) a protein consisting of the amino acid sequence as shown in SEQ ID NO: 6;
(b) a protein consisting of an amino acid sequence derived from the amino acid sequence as shown in SEQ ID NO: 6 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 6 and having activity of lysing the cell wall of Bacillus subtilis.

6. The lytic enzyme inhibitor according to claim 3, wherein the CwlS protein is any of the proteins (a) to (c) below:
(a) a protein consisting of the amino acid sequence as shown in SEQ ID NO: 55;
(b) a protein consisting of an amino acid sequence derived from the amino acid sequence as shown in SEQ ID NO: 55 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 55 and having activity of lysing the cell wall of Bacillus subtilis.

7. The lytic enzyme inhibitor according to claim 3, wherein the CwlO protein is any of the proteins (a) to (c) below;
(a) a protein consisting of the amino acid sequence as shown in SEQ ID NO: 57;
(b) a protein consisting of an amino acid sequence derived from the amino acid sequence as shown in SEQ ID NO: 57 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 57 and having activity of lysing the cell wall of Bacillus subtilis.

8. The lytic enzyme inhibitor according to claim 3, wherein the YddH protein is any of the proteins (a) to (c) below:
(a) a protein consisting of the amino acid sequence as shown in SEQ ID NO: 59;
(b) a protein consisting of an amino acid sequence derived from the amino acid sequence as shown in SEQ ID NO: 59 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 59 and having activity of lysing the cell wall of Bacillus subtilis.

9. A lysis inhibitor composed mainly of the Bacillus subtilis-derived YoeB gene or the YoeB protein encoded by a gene homologous to the YoeB gene.

10. The lysis inhibitor according to claim 9, wherein the YoeB protein is any of the proteins (a) to (c) below:
(a) a protein consisting of the amino acid sequence as shown in SEQ ID NO: 2;
(b) a protein consisting of an amino acid sequence derived from the amino acid sequence as shown in SEQ ID NO: 2 by substitution, deletion, addition, or insertion of one or several amino acids and having activity of binding to the Bacillus subtilis-derived lytic enzyme to inhibit lytic activity thereof; and
(c) a protein consisting of an amino acid sequence having 70% or higher homology to the amino acid sequence as shown in SEQ ID NO: 2 and having activity of binding to the Bacillus subtilis-derived lytic enzyme to inhibit lytic activity thereof.

11. The lysis inhibitor according to claim 9 or 10, which inhibits lysis of Bacillus subtilis.

12. The lysis inhibitor according to claim 11, wherein the Bacillus subtilis is a mutant that lacks at least one type of secretory protease.

13. An inhibitor of poly-γ-glutamic acid degradation composed mainly of the Bacillus subtilis-derived YoeB gene or the YoeB protein encoded by a gene homologous to the YoeB gene.

14. The inhibitor of poly-γ-glutamic acid degradation according to claim 13, wherein the YoeB protein is any of the proteins (a) to (c) below:
(a) a protein consisting of the amino acid sequence as shown in SEQ ID NO: 2;
(b) a protein consisting of an amino acid sequence derived from the amino acid sequence as shown in SEQ ID NO: 2 by substitution, deletion, addition, or insertion of one or several amino acids and having activity of binding to the Bacillus subtilis-derived lytic enzyme to inhibit lytic activity thereof; and
(c) a protein consisting of an amino acid sequence having 70% or higher homology to the amino acid sequence as shown in SEQ ID NO: 2 and having activity of binding to the Bacillus subtilis-derived lytic enzyme to inhibit lytic activity thereof.

15. The inhibitor of poly-γ-glutamic acid degradation according to claim 13 or 14, wherein the YoeB protein inhibits lytic activity of at least 1 lytic enzyme selected from the group consisting of the Bacillus subtilis-derived CwlE, CwlF, CwlS, and CwlO proteins and proteins functionally equivalent to said proteins to inhibit degradation of poly-γ-glutamic acid.

16. The inhibitor of poly-γ-glutamic acid degradation according to claim 15, wherein the CwlE protein is any of proteins (a) to (c) below:
(a) a protein consisting of the amino acid sequence as shown in SEQ ID NO: 4;
(b) a protein consisting of an amino acid sequence derived from the amino acid sequence as shown in SEQ ID NO: 4 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 4 and having activity of lysing the cell wall of Bacillus subtilis.

17. The inhibitor of poly-γ-glutamic acid degradation according to claim 15, wherein the CwlF protein is any of proteins (a) to (c) below:
(a) a protein consisting of the amino acid sequence as shown in SEQ ID NO: 6;
(b) a protein consisting of an amino acid sequence derived from the amino acid sequence as shown in SEQ ID NO: 6 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 6 and having activity of lysing the cell wall of Bacillus subtilis.

18. The inhibitor of poly-γ-glutamic acid degradation according to claim 15, wherein the CwlS protein is any of proteins (a) to (c) below:
(a) a protein consisting of the amino acid sequence as shown in SEQ ID NO: 55;
(b) a protein consisting of an amino acid sequence derived from the amino acid sequence as shown in SEQ ID NO: 55 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 55 and having activity of lysing the cell wall of Bacillus subtilis.

19. The inhibitor of poly-γ-glutamic acid degradation according to claim 15, wherein the CwlO protein is any of proteins (a) to (c) below:
(a) a protein consisting of the amino acid sequence as shown in SEQ ID NO: 57;
(b) a protein consisting of an amino acid sequence derived from the amino acid sequence as shown in SEQ ID NO: 57 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 57 and having activity of lysing the cell wall of Bacillus subtilis.

20. A microorganism whose lysis is inhibited by expressing the Bacillus subtilis-derived YoeB gene or a gene homologous to the YoeB gene under the control of an expression-inducible promoter.

21. The microorganism according to claim 20, wherein the YoeB gene encodes any of proteins (a) to (c) below:
(a) a protein consisting of the amino acid sequence as shown in SEQ ID NO: 2;
(b) a protein consisting of an amino acid sequence derived from the amino acid sequence as shown in SEQ ID NO: 2 by substitution, deletion, addition, or insertion of one or several amino acids and having activity of binding to the Bacillus subtilis-derived lytic enzyme to inhibit lytic activity thereof; and
(c) a protein consisting of an amino acid sequence having 70% or higher homology to the amino acid sequence as shown in SEQ ID NO: 2 and having activity of binding to the Bacillus subtilis-derived lytic enzyme to inhibit lytic activity thereof.

22. The microorganism according to claim 20 or 21, which lacks at least 1 secretory protease.

23. The microorganism according to any 1 of claims 20 to 22, which is a Bacillus subtilis-derived strain.

24. The microorganism according to any 1 of claims 20 to 23, which is capable of producing poly-γ-glutamic acid.

25. The microorganism according to claim 24, wherein the YoeB gene or the YoeB protein encoded by a gene homologous to the YoeB gene inhibits lytic activity of at least 1 lytic enzyme selected from the group consisting of the Bacillus subtilis-derived CwlE, CwlF, CwlS, and CwlO proteins and proteins functionally equivalent to said proteins to inhibit reduction of the molecular weight of poly-γ-glutamic acid.

26. The microorganism according to claim 25, wherein the CwlE protein is any of proteins (a) to (c) below:
(a) a protein consisting of the amino acid sequence as shown in SEQ ID NO: 4;
(b) a protein consisting of an amino acid sequence derived from the amino acid sequence as shown in SEQ ID NO: 4 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 4 and having activity of lysing the cell wall of Bacillus subtilis.

27. The microorganism according to claim 25, wherein the CwlF protein is any of proteins (a) to (c) below:
(a) a protein consisting of the amino acid sequence as shown in SEQ ID NO: 6;
(b) a protein consisting of an amino acid sequence derived from the amino acid sequence as shown in SEQ ID NO: 6 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 6 and having activity of lysing the cell wall of Bacillus subtilis.

28. The microorganism according to claim 25, wherein the CwlS protein is any of proteins (a) to (c) below:
(a) a protein consisting of the amino acid sequence as shown in SEQ ID NO: 55;
(b) a protein consisting of an amino acid sequence derived from the amino acid sequence as shown in SEQ ID NO: 55 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 55 and having activity of lysing the cell wall of Bacillus subtilis.

29. The microorganism according to claim 25, wherein the CwlO protein is any of proteins (a) to (c) below:
(a) a protein consisting of the amino acid sequence as shown in SEQ ID NO: 57;
(b) a protein consisting of an amino acid sequence derived from the amino acid sequence as shown in SEQ ID NO: 57 by substitution, deletion, addition, or insertion of one or several amino acids and having activity of lysing the cell wall of Bacillus subtilis; and
(c) a protein consisting of an amino acid sequence having 70% or higher homology to the amino acid sequence as shown in SEQ ID NO: 57 and having activity of lysing the cell wall of Bacillus subtilis.

30. A method for producing poly-γ-glutamic acid consisting of culturing a microorganism capable of producing poly-γ-glutamic acid in the presence of the Bacillus subtilis-derived YoeB gene or the YoeB protein encoded by a gene homologous to the YoeB gene and recovering the poly-γ-glutamic acid produced in the medium.

31. The method for producing poly-γ-glutamic acid according to claim 30, wherein the poly-γ-glutamic acid has a higher molecular weight than the poly-γ-glutamic acid produced in the absence of the YoeB protein.

32. The method for producing poly-γ-glutamic acid according to claim 30 or 31, wherein the microorganism is the microorganism according to any one of claims 24 to 29.

33. The method for producing poly-γ-glutamic acid according to any one of claims 30 to 32, wherein the YoeB protein is added to the medium.
